(19) United States
(12) Patent Application Publication
(10) Pub. No.: US 2010/0056555 A1
(43) Pub. Date: Mar. 4, 2010

(54) METHOD OF TREATING RAS ASSOCIATED CANCER

(75) Inventors: Ivan HORAK, West Orange, NJ (US); Puja SAPRA, Edison, NJ (US)

Correspondence Address: LUCAS & MERCANTI, LLP, 475 PARK AVENUE SOUTH, 15TH FLOOR, NEW YORK, NY, 10016, US

(73) Assignee: ENZON PHARMACEUTICALS, INC., Bridgewater, NJ (US)

(21) Appl. No.: 12/549,679

(22) Filed: Aug. 28, 2009

Related U.S. Application Data
(60) Provisional application No. 61/093,044, filed on Aug. 29, 2008.

(51) Int. Cl. A61K 31/4745; A61P 35/00; A61P 35/02; A61P 35/04; C12Q 1/68

(52) U.S. Cl. 514/283; 435/6

(57) ABSTRACT

New methods of treating Ras associated cancer, and especially for treating cancer that is resistant or refractory to other treatment methods, including resistant or refractory to treatment with C225 and/or CPT-11, are provided. The new methods employ PEG-conjugated 7-ethyl-10-hydroxycampothecin, alone, or in combination with other art-known anticancer agents or modalities.

### CROSS-REFERENCE TO RELATED APPLICATION

This application claims the benefit of priority from U.S. Provisional Patent Application Ser. No. 61/093,044, filed Aug. 29, 2008, the contents of which are incorporated herein by reference.

### FIELD OF THE INVENTION

The present invention relates to methods for treating Ras associated cancer by administering multi-arm polymeric prodrugs of 7-ethyl-10-hydroxycamptothecin. In particular, the invention relates to four-arm polyethylene glycol conjugates of 7-ethyl-10-hydroxycamptothecin and use thereof in treating cancer exhibiting an activated RAS gene.

### BACKGROUND OF THE INVENTION

In the field of oncology and cancer chemotherapy, there is a longstanding need for chemotherapeutic treatment methods that provide the optimal patient survival. One way this can be achieved is by identifying particular cancer types in a patient population that respond best to particular anticancer treatments, and thereby maximizing survival for such an identified patient population.

Mutated forms of RAS genes, termed oncogenes, have been implicated as causative agents in cancer. Both the normal or wild-type cellular RAS genes, and the oncogenes, encode a group of plasma membrane-associated G-proteins that bind guanine nucleotides with high affinity, and activate several downstream effector proteins including raf-1, P13-K, etc. These are known to activate several distinct signalling cascades that are involved in the regulation of cellular survival, proliferation and differentiation in response to extracellular stimuli, such as growth factors or hormones. The “classical” p21 ras family of mammalian proto-oncogenes consisting of Harvey-Ras (Hras, Ha-ras or HRAS), Kirsten-ras (Ki-Ras or K-Ras or KRAS) and Neuroblastoma-ras (N-Ras or NRAS) are the most well known members of the rapidly expanding Ras super-family of small GTPases. Due to alternative splicing, the KRAS gene can express either Kras2a or Kras2b proteins. These same alternative proteins are also art-known as Kras4a and Kras4b, respectively. The Kras 2a/2b nomenclature will be used herein to describe the Kras proteins, for consistency.

These three Ras family members are closely related, sharing 85% amino acid sequence homology, and function in very similar ways.

RAS oncogenes, and their normal cellular counterparts (a/k/a “wild type”), have been cloned and sequenced from a variety of species. Comparison of the structure of the wild type and mutated genes has revealed that they differ by point mutations that alter the amino acid sequence of the p21 protein. Naturally occurring mutations in the RAS oncogenes have been identified in codons that encode amino acids 10, 12, 13, 59, 61, etc. The most frequently observed mutation which converts a normal cellular RAS gene into its oncogenic counterpart is a mutation which leads to a substitution of glycine at position 12 of the protein by any other amino acid residue, with the exception of proline. Transforming activity is also observed if glycine is deleted, or if amino acids are inserted between alanine at position 11 and glycine at position 12.

Several in vitro (and in vivo) studies have demonstrated that the RAS family of proto-oncogenes are involved in the induction of malignant transformation; see, for example, Chin et al., 1999, Nature 400, 468-472. Consequently, the p21 Ras family proteins are regarded as important targets in development of anticancer drugs. It has been found that the Ras proteins are either over-expressed or mutated (often leading to constitutively active Ras proteins) in approximately 25% of all human cancers. See, for example, Schubert, et al. 2007, Nature Reviews, Cancer 7: 295-308, and Rajalingam, et al., 2007, Biochimica et Biophysica Acta 1773: 1177-1195, incorporated by reference herein in their entireties. Schubert et al. used data from the Sanger Institute Catalog of Somatic Mutations (see website at http://www.sanger.ac.uk/), incorporated by reference herein, to tabulate the incidence of human Ras-associated cancers by type. Schubert et al. reports that KRAS mutations are present in a substantial percentage of cancers. For example, 33% of biliary tract cancers are KRAS mutation positive; 32% of colon cancers are KRAS mutation positive, and up to 60% of pancreatic cancers are KRAS mutation positive. Shubert et al. reports that other RAS mutations are somewhat less frequently associated with cancers, but the numbers are still significant, e.g., 18% of melanomas are NRAS mutation positive. The reader is referred to Schubert et al., and particularly Table 2 of that article, incorporated by reference herein, for further background on the incidence of RAS mutations in cancer.

Interestingly, the RAS gene mutations in most cancer types are frequently limited to only one of the RAS genes and are dependent on tumor type and tissue.

Because of the evidence of Ras involvement in cancer development, interruption of the Ras pathway has been a major focus for drug development, as described, for example, by Adjei, 2001, J. National Cancer Institute, 93 (14):1062-1074, incorporated by reference herein. Thus, previous efforts have concentrated on either inhibiting ras maturation and thereby membrane localization, or inhibiting Ras protein expression.

Camptothecin is a water-insoluble cytotoxic alkaloid produced by Camptotheca accuminata trees indigenous to China and Nothapodytes foetida trees indigenous to India. Camptothecin and related analogs are known to be potential anticancer agents and have demonstrated therapeutic activity in vitro and in vivo. Camptothecin and analogs are known as DNA topoisomerase I inhibitors. For example, one camptothecin analog is Irinotecan (CPT-11, Camptosar®) which is also a DNA topoisomerase I inhibitor, and has also showed anticancer activity. An active metabolite of CPT-11 is 7-ethyl-10-hydroxycampothecin.

According to the current medical practice, patients with advanced colorectal cancer will receive a regimen that includes, e.g., irenotecan (Camptosar®) or oxaliplatin (Eloxatin®) as the first or second line therapy. Patients who are resistant or refractory to previous therapies will receive a combination of Camptosar® in combination with EGFR1 targeted therapy (e.g., Erbitux®). Patients with Kras mutation are refractory to this combination therapy and therefore there is a high unmet medical need to develop alternative therapy for this patient population. Furthermore, patients with Kras mutated tumors other than colorectal cancer may benefit from this novel therapy as well. There remains a longstanding need in the art for further improved anticancer therapies, and particularly for new and improved method of treating Ras mutation positive cancers or Ras associated cancers.

### SUMMARY OF THE INVENTION

In order to overcome the above problems and improve the treatment of Ras associated cancers, and in particular, resistant or refractory CPT-11/EGFR1 antagonist treated Ras associated cancers, there are provided new methods to treat such Ras associated cancers by administering PEG-conjugated 7-ethyl-10-hydroxycampothecin, alone or in combination with other art-known anticancer agents or modalities.

Broadly, the invention provides a method of treating a Ras-associated cancer in mammals comprising:

(i) determining the presence of a mutation in a RAS gene in a cancer in a mammal, for example, a human, having cancer; and

(ii) administering an effective amount of a compound of Formula I:

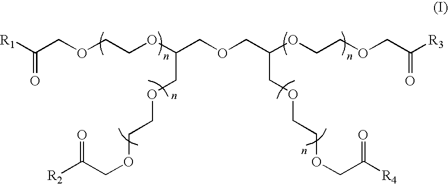

wherein

R₁, R₂, R₃ and R₄ are independently OH or

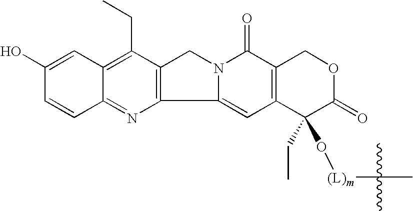

wherein
L is a bifunctional linker, and each L is the same or different when (m) is equal to or greater than 2;
(m) is 0 or a positive integer; and
(n) is a positive integer;
provided that R₁, R₂, R₃ and R₄ are not all OH,
   or a pharmaceutically acceptable salt thereof;
   to a mammal having said mutation in said RAS gene.

In one aspect, L is an amino acid or amino acid derivative and the amino acid derivative is chosen from 2-aminoadipic acid, 3-aminoadipic acid, beta-alanine, beta-aminopropionic acid, 2-aminobutyric acid, 4-aminobutyric acid, piperidinic acid, 6-aminocaproic acid, 2-aminoheptanoic acid, 2-aminoisobutyric acid, 3-aminoisobutyric acid, 2-aminopimelic acid, 2,4-aminobutyric acid, desmosine, 2,2-diaminopimelic acid, 2,3-diaminopropionic acid, N-ethylglycine, N-ethylasparagine, 3-hydroxyproline, 4-hydroxyproline, isodesmosine, allo-isoleucine, N-methylglycine, sarcosine, N-methylisoleucine, N-methyllysine, N-methylvaline, norvaline, norleucine, and ornithine. In another aspect, L is glycine, alanine, methionine or sarcosine, and most preferably glycine.

Alternatively, L is one selected from one of the following:

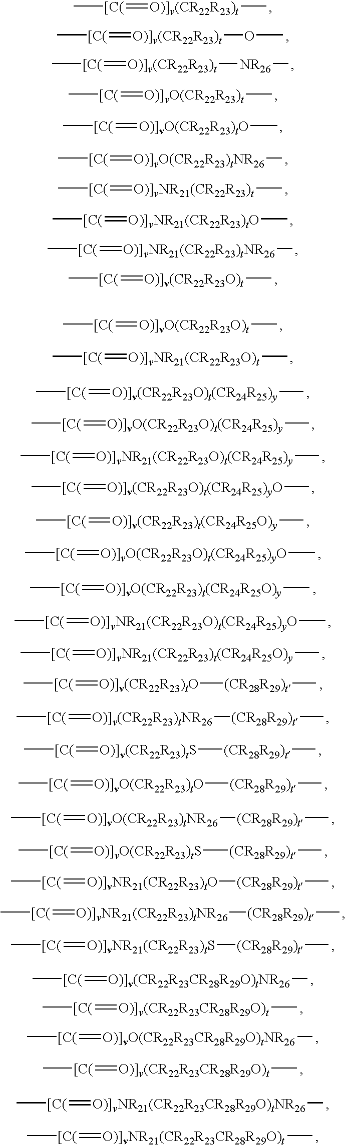

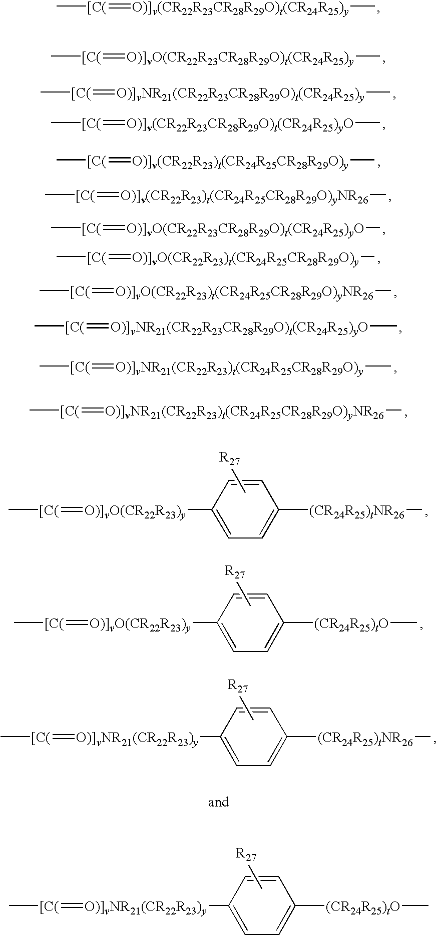

wherein:

R₂₁-R₂₉ are independently selected from the group consisting of hydrogen, amino, substituted amino, azido, carboxy, cyano, halo, hydroxyl, nitro, silyl ether, sulfonyl, mercapto, C₁₋₆ alkylmercapto, arylmercapto, substituted arylmercapto, substituted C₁₋₆ alkylthio, C₁₋₆ alkyls, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, and substituted arylcarbonyloxy;

(t), (t′) and (y) are independently zero or a positive integer; and

(v) is 0 or 1.

The above described method of the invention can be conducted with a compound of Formula I, wherein (m) is from about 1 to about 10; (n) is from about 28 to about 341; or alternatively, (n) is from about 114 to about 239, and preferably, (n) is about 239.

In a further aspect, the above provided method according to the invention is conducted wherein the compound of Formula (I) is part of a pharmaceutical composition, and R₁, R₂, R₃ and R₄ are all:

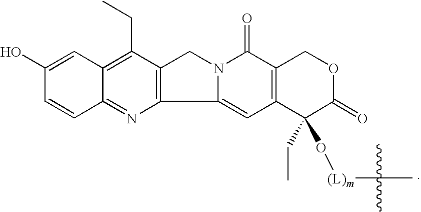

In yet a further aspect, the above provided method according to the invention is conducted wherein the compound of Formula (I) is chosen from:

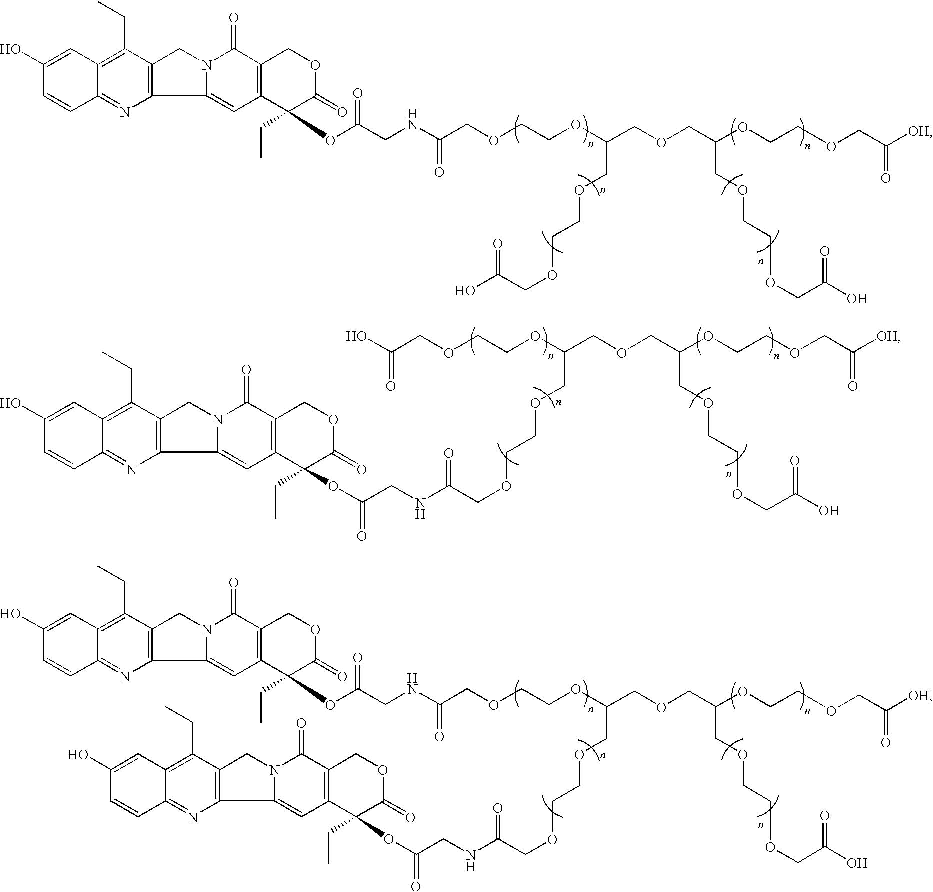

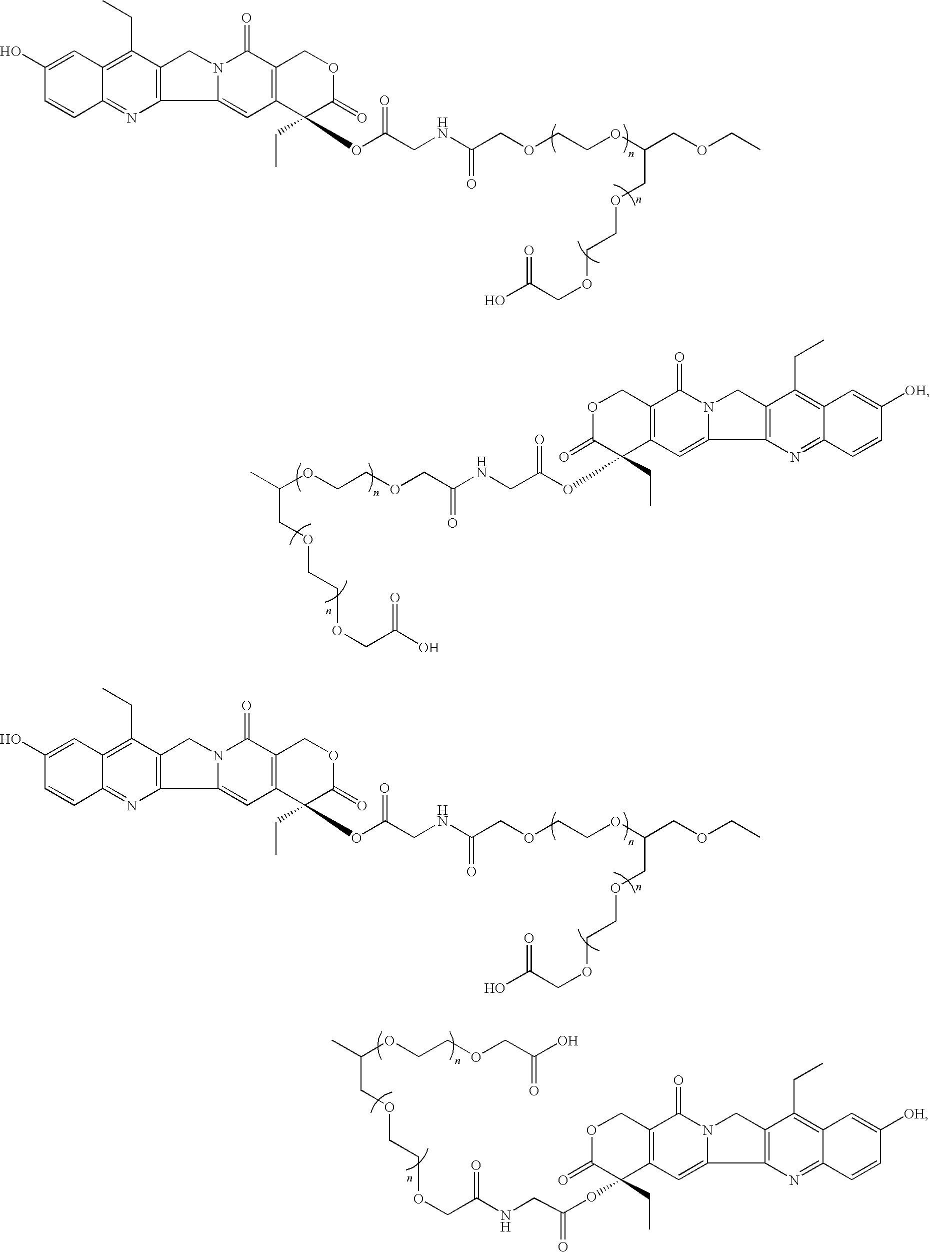

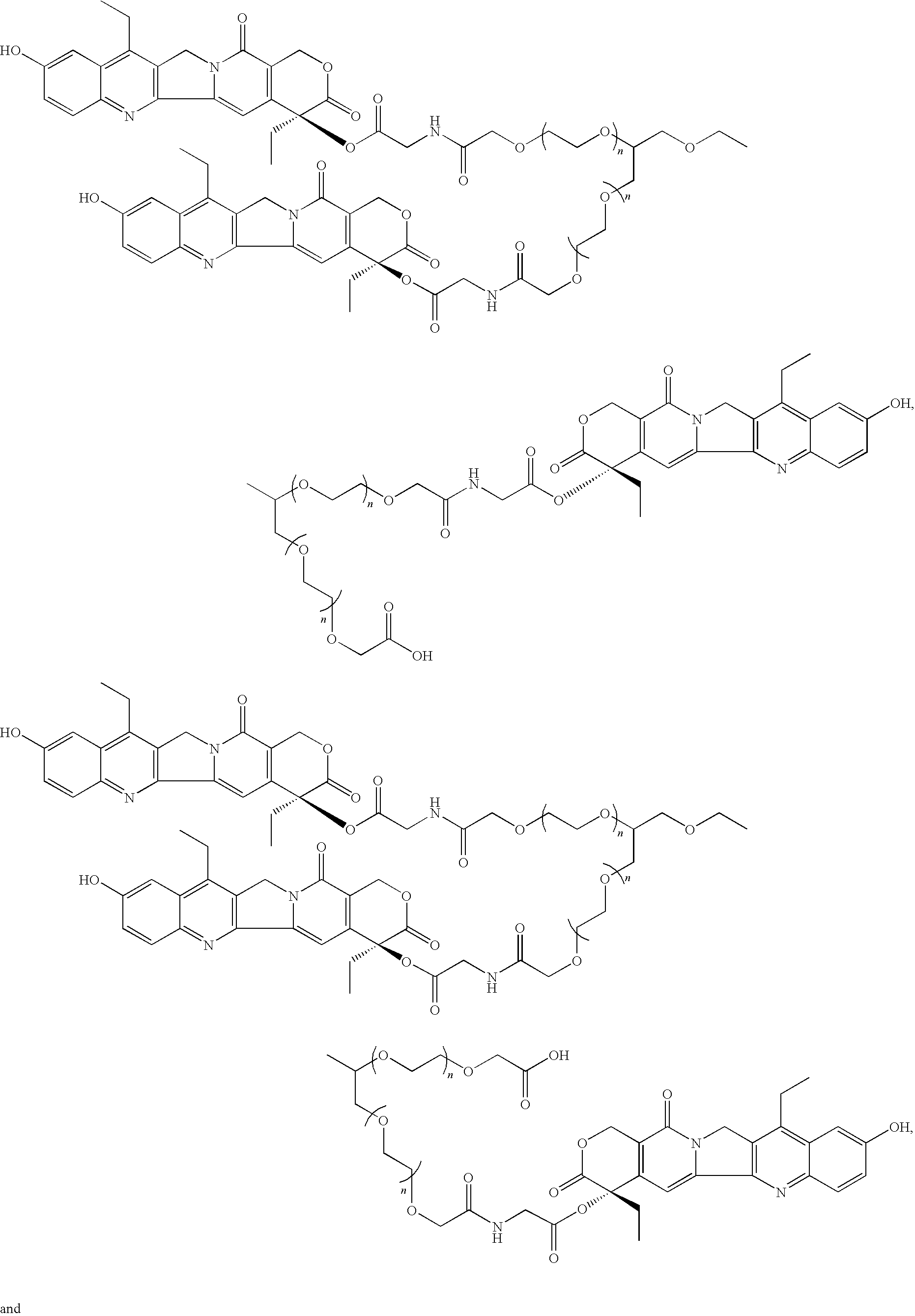

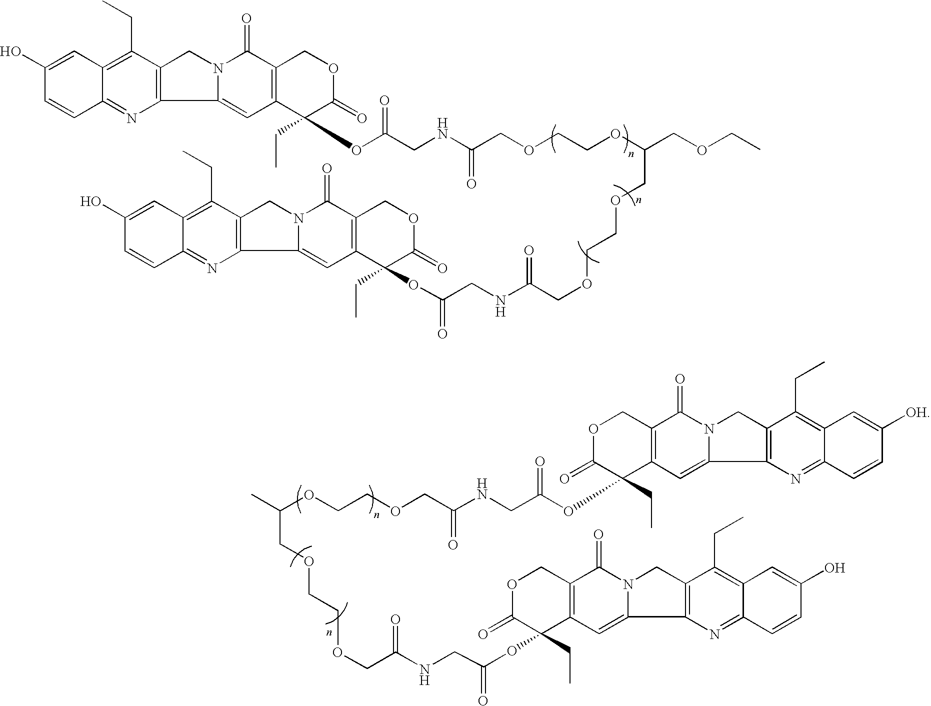

In particular, the above provided method according to the invention is conducted wherein the compound of Formula (I)

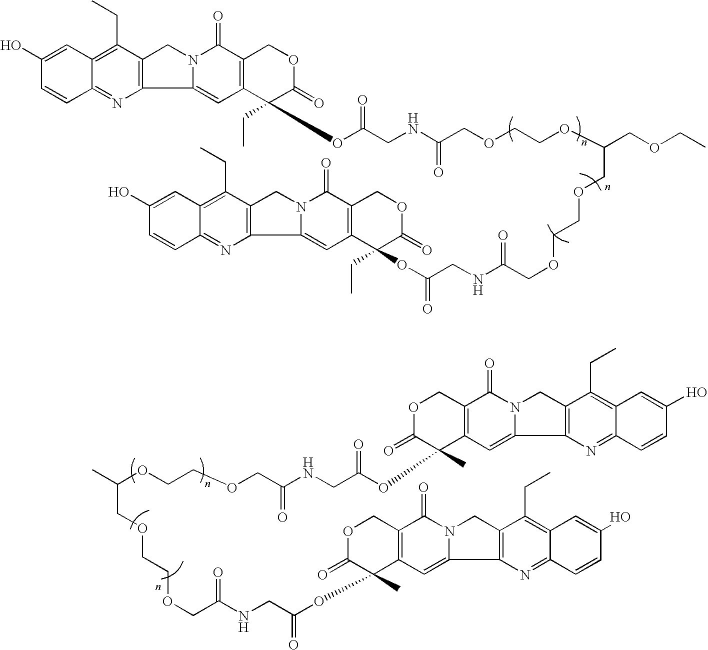

In a still further aspect, the above provided method of the invention is conducted wherein said determining of the presence of the mutation in the RAS gene in the mammal having cancer comprises the steps of:

comparing nucleic acids encoding a Ras protein isolated from the mammal to nucleic acids encoding a wild-type Ras protein.

Optionally, the Ras protein is chosen from a Kras protein, a Hras protein, and a Nras protein. For example, the Kras protein is Kras2b or Kras2a,

Further still, the above provided method of the invention is conducted wherein the mutation in the RAS gene is identified in a nucleic acid region encoding amino acids 1 to 165 of SEQ ID NO: 1, SEQ ID NO: 2, SEQ ID NO: 3 or SEQ ID NO: 4. The mutation in the expressed Ras protein includes, for example, an amino acid substitution or insertion at one or more amino acid positions of 10, 12, 13, 59, 61, 117, 146 or a combination thereof, relative to SEQ ID NO: 1 or 2. For example, the method of determining the presence of the mutation in the gene encoding a Ras protein in the cancer comprises detecting single nucleotide polymorphisms (“SNPs”) associated with a known mutant RAS gene in said cancer.

In an additional aspect, the above provided method of the invention is conducted wherein the mammal having the mutation in the RAS gene has a cancer chosen from solid tumors, colorectal cancer, pancreatic cancer, lung cancer, small cell lung cancer, stomach cancer, lymphomas, acute lymphocytic leukemia (ALL), melanoma, acute myeloid leukemia (AML), breast cancer, bladder cancer, glioblastoma, ovarian cancer, non-Hodgkin's lymphoma, anal cancer, head and neck cancer. The cancer can be, for example, a metastatic cancer and is preferably a cancer that is resistant or refractory to an anti-cancer agent that is not a compound of Formula I. For example, the cancer is resistant to an anti-cancer agent that is chosen from camptothecin, CPT-11, an epidermal growth factor receptor antagonist, e.g., cetuximab, and combinations thereof.

Simply by way of example, the above provided method of the invention is conducted wherein the compound of Formula I is administered in amounts of from about 0.5 mg/m² body surface/dose to about 50 mg/m² body surface/dose, and more particularly, wherein the compound of Formula I is administered in amounts of from about 1 mg/m² body surface/dose to about 18 mg/m² body surface/dose, and even more particularly, wherein the compound of Formula I is administered according to a protocol of from about 1.25 mg/m body surface/dose to about 16.5 mg/m² body surface/dose given weekly for three weeks, followed by 1 week without treatment. In certain aspects, the amount administered weekly is about 5 mg/m² body surface/dose.

In an alternative aspect, the invention provides a method of treating a Ras-associated cancer in a mammal, comprising administering an effective amount of a compound of Formula I:

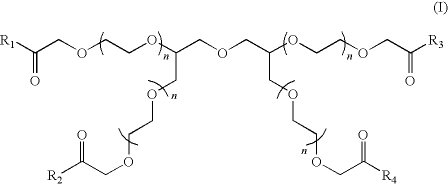

wherein

R₁, R₂, R₃ and R₄ are independently OH or

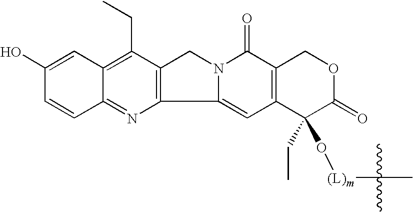

wherein
L is a bifunctional linker, and each L is the same or different when (m) is equal to or greater than 2,
(m) is 0 or a positive integer; and
(n) is a positive integer;
provided that R₁, R₂, R₃ and R₄ are not all OH;

or a pharmaceutically acceptable salt thereof;

to a mammal, such as a human, having said mutation in a RAS protein,
wherein the cancer is a Ras associated cancer comprising an activated Ras protein.

In a still further aspect of the invention, the inventive method is conducted by administering a polymeric conjugate of a camptothecin to a mammal having a Ras positive cancer. The polymeric conjugate includes a compound of Formula (II) or (III):

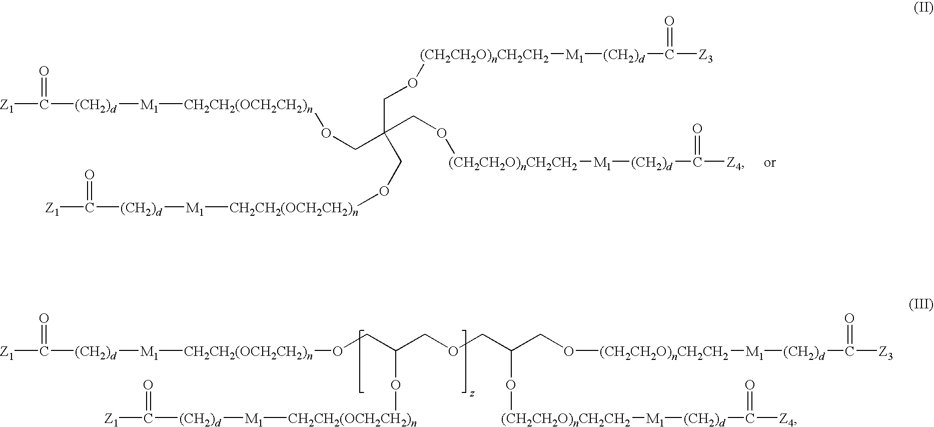

wherein

Z₁, Z₂, Z₃ and Z₄ are independently OH or -(L)ₘ-D;

L is a bifunctional linker;

D is 7-ethyl-10-hydroxycamptothecin;

M₁ is O, S, or NH;

(d) is zero or a positive integer of from about 1 to about 10;

(z) is zero or a positive integer of from 1 to about 29;

(m) is 0 or a positive integer, wherein each L is the same or different when (m) is equal to or greater than 2; and

(n) is a positive integer of from about 10 to about 2,300 so that the polymeric portion of the compound has the total number average molecular weight of from about 2,000 to about 100,000 daltons,

provided that Z₁, Z₂, Z₃ and Z₄ are not all OH.

Advantages will be apparent from the following description and drawings.

### BRIEF DESCRIPTION OF THE DRAWINGS

FIG. 1 schematically illustrates a reaction scheme for preparing four-arm polyethylene glycol acids.

FIG. 2 schematically illustrates a reaction scheme for preparing 4arm-PEG-Gly-(7-ethyl-10-hydroxycamptothecin) described in Examples 3-7.

FIG. 3 schematically illustrates a reaction scheme for preparing 4arm-PEG-Ala-(7-ethyl-10-hydroxycamptothecin) described in Examples 8-12.

FIG. 4 schematically illustrates a reaction scheme for preparing 4arm-PEG-Met-(7-ethyl-10-hydroxycamptothecin) described in Examples 13-16.

FIG. 5 schematically illustrates a reaction scheme for preparing 4arm-PEG-Sar-(7-ethyl-10-hydroxycamptothecin) described in Examples 17-21.

FIG. 6 shows the stability of 4arm-PEG-Gly-(7-ethyl-10-hydroxycamptothecin) as described in Example 23. “PBS” indicates phosphate buffered saline.

FIG. 7 shows effect of pH on stability of 4arm-PEG-Gly-(7-ethyl-10-hydroxycamptothecin) as described in Example 24.

FIG. 8A shows pharmacokinetic profiles of compound 9 as described in Example 25.

FIG. 8B shows pharmacokinetic profiles of 4arm-PEG-Gly-(7-ethyl-10-hydroxy-camptothecin) released from compound 9, as described in Example 25.

FIG. 9 shows the application of compound 9, CPT-11 or Erbitux® (C225) in a KRAS mutated xenograft model of human lung cancer (Calu 6), as described in Example 26. (▪) indicates saline; compound 9 at 30 mg/kg/qd×1 (▴) (this curve is behind the (Δ) curve); CPT-11 at 80 mg/kg qd×1 (4); compound 9 at 10 mg/kg q2d×5 (Δ); CPT-11 at 40 mg/kg q2d×5 (⋄); () indicates C-225.

FIG. 10 shows the effects of compound 9, CPT-11 in a KRAS xenograft model of human lung cancer (Calu 6) that has failed C225 treatment, as described in Example 27. (▪) indicates saline; () indicates C-225; (Δ) indicates compound 9 at 10 mg/kg q2d×5 with C-225 challenge; (⋄) indicates CPT-11 at 40 mg/kg q2d×5 with C-225 challenge.

FIG. 11A describes that the efficacy of compound 9 was evaluated in a KRAS mutant pancreatic xenograft model (MiaPaCa-2) with a single injection. Saline (▪), compound 9 at MTD (▴), CPT-11 at MTD (◯), or CPT-11 at equivalent MTD of compound 9 (). See Sapra et al. Mar. 15, 2008, Clin Cancer Res 14(6):1888-1896, incorporated by reference herein.

FIG. 11B describes that the efficacy of compound 9 was evaluated in a Kras mutant pancreatic xenograft model (MiaPaCa-2) with multiple (q2d×5) injections. Saline (▪), compound 9 at MTD (▴), CPT-11 at MTD (◯), or CPT-11 at equivalent MID of compound 9 (). See Sapra et al. Mar. 15, 2008, Clin Cancer Res 14(6):1888-1896, incorporated by reference herein.

FIG. 12 illustrates the effect of compound 9, CPT-11 or Erbitux (C-225) in a Kras mutated xenograft model of human colorectal (SW480) is evaluated. Saline control (▪); () indicates C-225; (Δ) indicates compound 9 at 10 mg/kg q2d×5; (⋄) indicates CPT-11 at 40 mg/kg q2d×5 with C-225 challenge; (▴) indicates compound 9 at 30 mg/kg q2d×5; (Δ) indicates compound 9 at 10 mg/kg q2d×5; (◯) indicates compound 9 at 1 mg/kg q2d×5; and (♦) indicates CPT-11 at 80 mg/kg qd×1.

FIG. 13 illustrates the effect of compound 9 and CPT-11 in Kras mutant xenografts that failed in treatment with C225. Saline control (▪); (⋄) indicates CPT-11 at 40 mg/kg day 21, q2d×7 with C-225 challenge; (Δ) indicates compound 9 at 10 mg/kg q2d×7 with C-225 challenge; () indicates C-225 with C-225 challenge.

FIG. 14. Illustrates the effect of compound 9 on HCT-116 K ras mutant human colorectal xenografts. Saline control (▪); and (▴) indicates compound 9 at 10 mg/kg.

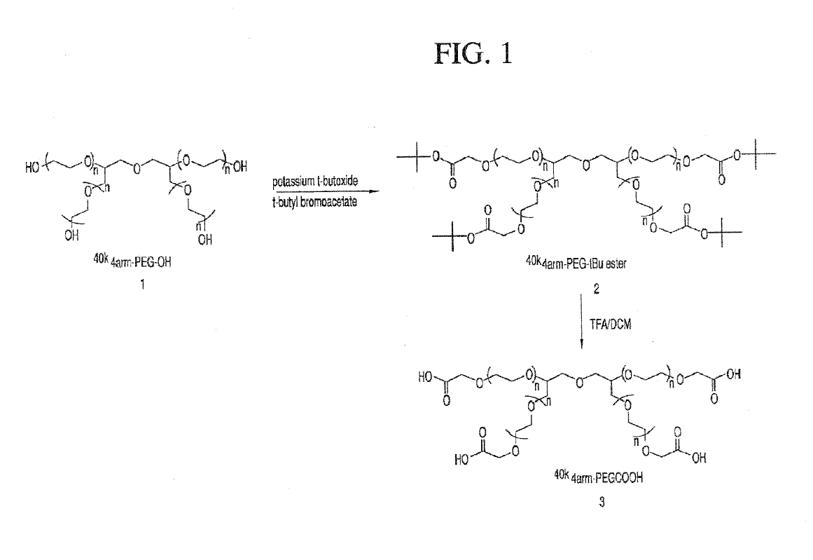

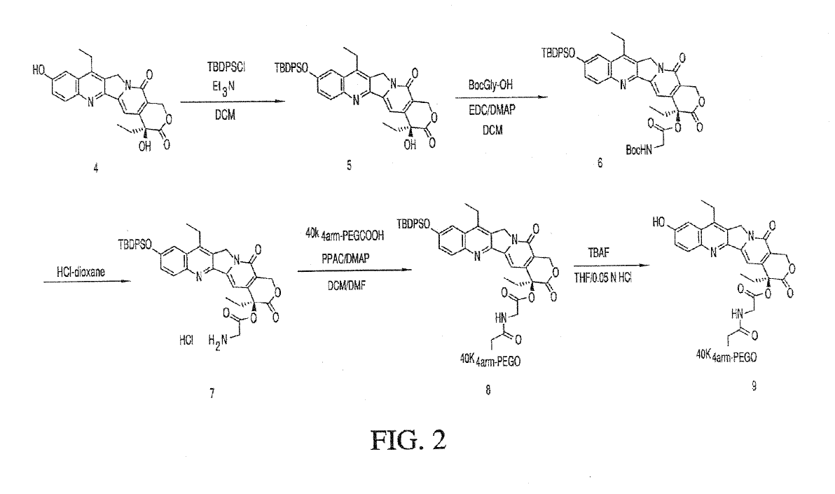

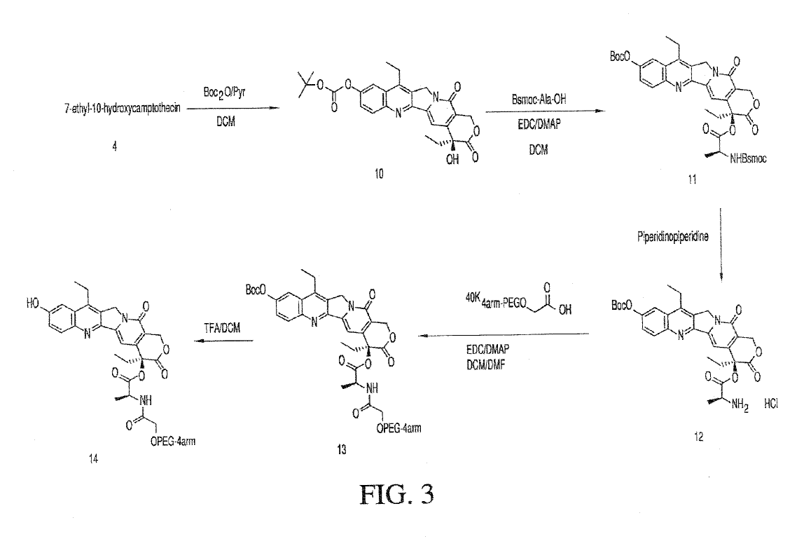

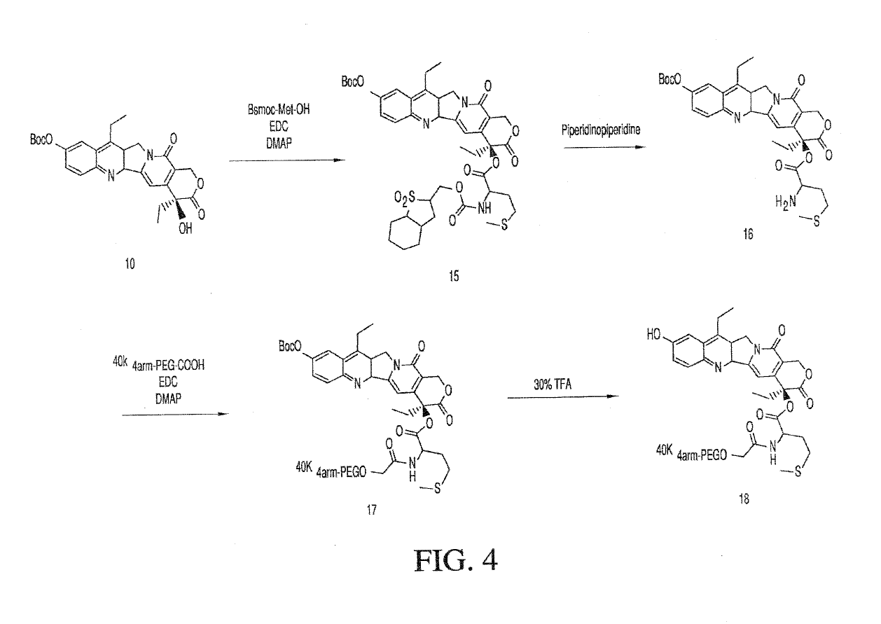

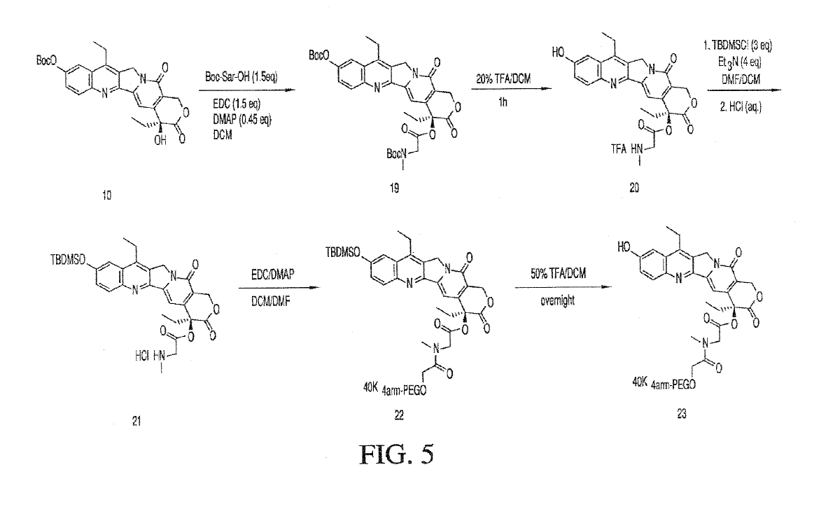

### DETAILED DESCRIPTION OF THE INVENTION

### A. Overview

Accordingly, in one aspect of the present invention, there are provided methods of treating Ras associated cancer in a mammal, e.g., a human. The method includes:

(i) determining the presence of a mutation in a RAS gene in a cancer, in a mammal having cancer; and

(ii) administering an effective amount of a compound of Formula I:

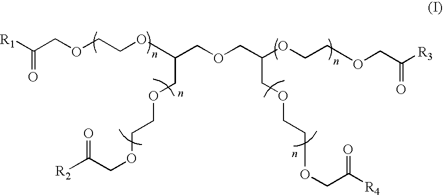

wherein

R₁, R₂, R₃ and R₄ are independently OH or

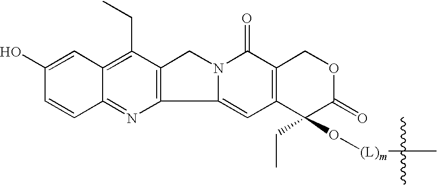

wherein
L is a bifunctional linker, and each L is the same or different when (m) is equal to or greater than 2;
(m) is 0 or a positive integer such as, for example, from about 1 to about 10 (for example, 1, 2, 3, 4, 5 or 6), and preferably 1; and
(n) is a positive integer, preferably from about 28 to about 341, more preferably from about 114 to about 239, yet more preferably about 239;
provided that R₁, R₂, R₃ and R₄ are not all OH,
   or a pharmaceutically acceptable salt thereof;
   to a mammal having said mutation in a RAS gene.

In one preferred embodiment, the method includes a compound of Formula (I) as part of a pharmaceutical composition, and R₁, R₂, R₃ and R₄ are all:

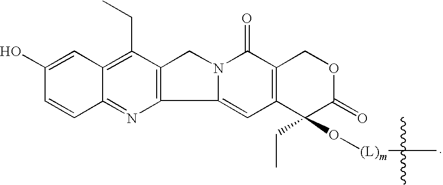

In another aspect of the invention, there are provided methods of treating a Ras associated cancer in a mammal. The method includes administering an effective amount of a compound of Formula I:

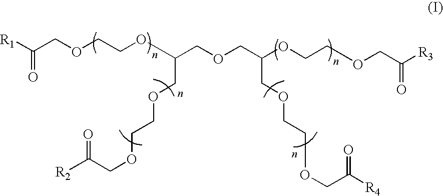

wherein

R₁, R₂, R₃ and R₄ are independently OH or

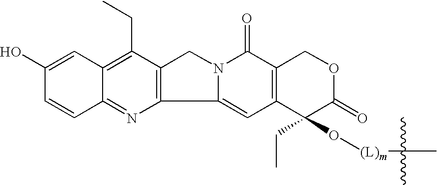

wherein
L is a bifunctional linker, and each L is the same or different when (m) is equal to or greater than 2;
(m) is 0 or a positive integer such as, for example, from about 1 to about 10 (for example, 1, 2, 3, 4, 5 or 6), and preferably 1; and
(n) is a positive integer, preferably from about 28 to about 341, more preferably from about 114 to about 239, yet more preferably about 239;
provided that R₁, R₂, R₃ and R₄ are not all OH,

or a pharmaceutically acceptable salt thereof;

to a mammal having said mutation in a Ras protein,
wherein the cancer is a Ras associated cancer having an activated Ras protein.

The following definitions provide greater clarity of description.

For purposes of the present invention, the terms “cancer” and “tumor” are used interchangeably, unless otherwise indicated. “Cancer” encompasses malignant and/or metastatic cancer, unless otherwise indicated. Further, a Ras associated cancer is understood to be a cancer or tumor expressing or otherwise including a mutated RAS gene resulting in activation of the RAS gene as a cancer gene or oncogene. A Ras associated cancer can also be a cancer or tumor that expresses a mutein Ras protein. Mutation of the KRAS gene, to produce mutein Kras protein(s), is the most frequent, occurring in approximately 85% of Ras-mutated cancers, while the NRAS gene is mutated in about 15% and HRAS gene is mutated in less than 1% of Ras-mutated cancers. In an alternative aspect, a Ras associated cancer can also be a cancer or tumor wherein there is aberrant ras signaling. Aberrant Ras signaling can, for example, also be activated by overexpression of growth factor receptors such as EGFR and amplification of upstream activators such as PI3K and Akt or mutation of inhibitors of ras activation such as PTEN. In a further alternative aspect, a Ras associated cancer can also be a cancer or tumor wherein there is an overexpression of the Ras protein or an abnormal level of activated Ras protein.

One aspect of the invention provides a method of treating Ras associated cancers that are resistant or refractory to conventional anticancer methods, including chemotherapy. In one particular aspect, the treatment is effective for cancers resistant or refractory to anti-EGFR agents, alone or in combination with camptothecin (CPT) or CPT-11 associated therapy. Alternatively, the present invention provides a method of treating cancers showing topoisomerase I mediated resistance or refractory phenomenon.

For purposes of the present invention, a resistant or refractory cancer which can be treated with the methods described herein includes a solid tumor, lymphomas, lung cancer, small cell lung cancer, acute lymphocytic leukemia (ALL), breast cancer, colorectal cancer, pancreatic cancer, glioblastoma, ovarian cancer and gastric cancer. The forgoing list is not meant to be exclusive and those of ordinary skill will, of course, realize that other resistant or refractory cancers not specifically mentioned herein are intended for inclusion.

For purposes of the present invention, the term “residue” shall be understood to mean that portion of a compound, to which it refers, e.g., camptothecin analog, 7-ethyl-10-hydroxycamptothecin, amino acid, etc. that remains after it has undergone a substitution reaction with another compound.

For purposes of the present invention, the term “polymeric containing residue” or “PEG residue” shall each be understood to mean that portion of the PEG which remains after it has undergone a reaction with, e.g., an amino acid, 7-ethyl-10-hydroxycamptothecin, or other suitable compound.

For purposes of the present invention, the term “alkyl” refers to a saturated aliphatic hydrocarbon, including straight-chain, branched-chain, and cyclic alkyl groups. The term “alkyl” also includes alkyl-thio-alkyl, alkoxyalkyl, cycloalkylalkyl, heterocycloalkyl, and C₁₋₆ alkylcarbonylalkyl groups. Preferably, the alkyl group has 1 to 12 carbons. More preferably, it is a lower alkyl of from about 1 to 7 carbons, yet more preferably about 1 to 4 carbons.

The alkyl group can be substituted or unsubstituted. When substituted, the substituted group(s) preferably include halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups.

For purposes of the present invention, the term “substituted” refers to adding or replacing one or more atoms contained within a functional group or compound with one of the moieties from the group of halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ alkylcarbonylalkyl, aryl, and amino groups.

For purposes of the present invention, the term “alkenyl” refers to groups containing at least one carbon-carbon double bond, including straight-chain, branched-chain, and cyclic groups. Preferably, the alkenyl group has about 2 to 12 carbons. More preferably, it is a lower alkenyl of from about 2 to 7 carbons, yet more preferably about 2 to 4 carbons. The group can be substituted or unsubstituted. When substituted the substituted group(s) include halo, oxy, azido, nitro, cyano, alkyl alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups.

For purposes of the present invention, the term “alkynyl” refers to groups containing at least one carbon-carbon triple bond, including straight-chain, branched-chain, and cyclic groups. Preferably, the alkynyl group has about 2 to 12 carbons. More preferably, it is a lower alkynyl of from about 2 to 7 carbons, yet more preferably about 2 to 4 carbons. The alkynyl group can be substituted or unsubstituted. When substituted the substituted group(s) include halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups. Examples of “alkynyl” include propargyl, propynyl, and 3-hexynyl.

For purposes of the present invention, the term “aryl” refers to an aromatic hydrocarbon ring system containing at least one aromatic ring. The aromatic ring can optionally be fused or otherwise attached to other aromatic hydrocarbon rings or non-aromatic hydrocarbon rings. Examples of aryl groups include, for example, phenyl, naphthyl, 1,2,3,4-tetrahydronaphthalene and biphenyl. Preferred examples of aryl groups include phenyl and naphthyl.

For purposes of the present invention, the term “cycloalkyl” refers to a C₃₋₈ cyclic hydrocarbon. Examples of cycloalkyl include cyclopropyl, cyclobutyl, cyclopentyl, cyclohexyl, cycloheptyl and cyclooctyl.

For purposes of the present invention, the term “cycloalkenyl” refers to a C₃₋₈ cyclic hydrocarbon containing at least one carbon-carbon double bond. Examples of cycloalkenyl include cyclopentenyl, cyclopentadienyl, cyclohexenyl, 1,3-cyclohexadienyl, cycloheptenyl, cycloheptatrienyl, and cyclooctenyl.

For purposes of the present invention, the term “cycloalkylalkyl” refers to an alklyl group substituted with a C₃₋₈ cycloalkyl group. Examples of cycloalkylalkyl groups include cyclopropylmethyl and cyclopentylethyl.

For purposes of the present invention, the term “alkoxy” refers to an alkyl group of indicated number of carbon atoms attached to the parent molecular moiety through an oxygen bridge. Examples of alkoxy groups include, for example, methoxy, ethoxy, propoxy and isopropoxy.

For purposes of the present invention, an “alkylaryl” group refers to an aryl group substituted with an alkyl group.

For purposes of the present invention, an “aralkyl” group refers to an alkyl group substituted with an aryl group.

For purposes of the present invention, the term “alkoxyalkyl” group refers to an alkyl group substituted with an alkloxy group.

For purposes of the present invention, the term “amino” refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

For purposes of the present invention, the term “halogen” or “halo” refers to fluoro, chloro, bromo, and iodo.

For purposes of the present invention, the term “heteroatom” refers to nitrogen, oxygen, and sulfur.

For purposes of the present invention, the term “heterocycloalkyl” refers to a non-aromatic ring system containing at least one heteroatom selected from nitrogen, oxygen, and sulfur. The heterocycloalkyl ring can be optionally fused to or otherwise attached to other heterocycloalkyl rings and/or non-aromatic hydrocarbon rings. Preferred heterocycloalkyl groups have from 3 to 7 members. Examples of heterocycloalkyl groups include, for example, piperazine, morpholine, piperidine, tetrahydrofuran, pyrrolidine, and pyrazole. Preferred heterocycloalkyl groups include piperidinyl, piperazinyl, morpholinyl, and pyrrolidinyl.

For purposes of the present invention, the term “heteroaryl” refers to an aromatic ring system containing at least one heteroatom selected from nitrogen, oxygen, and sulfur. The heteroaryl ring can be fused or otherwise attached to one or more heteroaryl rings, aromatic or non-aromatic hydrocarbon rings or heterocycloalkyl rings. Examples of heteroaryl groups include, for example, pyridine, furan, thiophene, 5,6,7,8-tetrahydroisoquinoline and pyrimidine. Preferred examples of heteroaryl groups include thienyl, benzothienyl, pyridyl, quinolyl, pyrazinyl, pyrimidyl, imidazolyl, benzimidazolyl, furanyl, benzofuranyl, thiazolyl, benzothiazolyl, isoxazolyl, oxadiazolyl, isothiazolyl, benzisothiazolyl, triazolyl, tetrazolyl, pyrrolyl, indolyl, pyrazolyl, and benzopyrazolyl.

In some embodiments, substituted alkyls include carboxyalkyls, aminoalkyls, dialkylaminos, hydroxyalkyls and mercaptoalkyls; substituted alkenyls include carboxyalkenyls, aminoalkenyls, dialkenylaminos, hydroxyalkenyls and mercaptoalkenyls; substituted alkynyls include carboxyalkynyls, aminoalkynyls, dialkynylaminos, hydroxyalkynyls and mercaptoalkynyls; substituted cycloalkyls include moieties such as 4-chlorocyclohexyl; aralkyls include moieties such as tolyl.

For purposes of the present invention, “positive integer” shall be understood to include an integer equal to or greater than 1 and as will be understood by those of ordinary skill to be within the realm of reasonableness by the artisan of ordinary skill.

For purposes of the present invention, the term “linked” shall be understood to include covalent (preferably) or noncovalent attachment of one group to another, i.e., as a result of a chemical reaction.

The terms “effective amounts” and “sufficient amounts” for purposes of the present invention shall mean an amount which achieves a desired effect or therapeutic effect as such effect is understood by those of ordinary skill in the art. An effective amount for each mammal or human patient to be treated is readily determined by the artisan in a range that provides a desired clinical response while avoiding undesirable effects that are inconsistent with good practice. Dose ranges are provided hereinbelow.

The RAS gene is a family of retrovirus-associated DNA sequences (ras) that were originally isolated from murine sarcoma viruses. The three cellar RAS genes encode four highly homologous 21 kD proteins: Hras, Nras, Kras2a and Kras2b. Kras2a and Kras2b result from alternative splicing at the C terminus. The Ras genes exist in a wild-type, or non-cancer promoting state. The wild-type Ras proteins are as follows: Kras2a (SEQ ID NO: 1); Kras2b (SEQ ID NO: 2); Hras (SEQ ID NO: 3) and Nras (SEQ ID NO: 4). The cDNAs expressing the respective proteins are as follows: KRAS (SEQ ID NO: 5), HRAS (SEQ ID NO: 6) and NRAS (SEQ ID NO: 7).

The N-terminal portion (residues 1-165) of HRAS, KRAS and NRAS comprise a highly conserved G domain that has a common structure. Ras proteins diverge substantially at the C-terminal end, which is known as the hypervariable region. This region contains residues that specify posttranslational protein modifications that are essential for targeting Ras proteins to the cytosolic leaflet of cellular membranes.

The somatic missense Ras mutations found in cancer cells introduce amino acid substitutions at positions 10, 12, 13, 59, 61, 117 and 146, and in most cases at positions 12, 13 and 61. The glycine to valine mutation at residue 12 renders the GTPase domain of Ras insensitive to inactivation by GAP and thus stuck in the “on state”. Ras requires a GAP for inactivation as it is a relatively poor catalyst on its own, as opposed to other G-domain-containing proteins such as the alpha subunit of heterotrimeric G proteins. Replacing glycine 12 of Ras with any other amino acid except proline also biochemically activates Ras. These substitutions are thought to be unfavourable in the GTP-GDP transition state because of a steric clash of side chains with the catalytic arginine and with the side chain of glutamine. Substituting proline for glycine 12 renders Ras resistant to GAPs, but has increased intrinsic GTP hydrolysis.

The three-dimensional structure at residue 61 is responsible for stabilizing the transition state for GTP hydrolysis. Because enzyme catalysis in general is achieved by lowering the energy barrier between substrate and product, mutation of Q61 necessarily reduces the rate of intrinsic Ras GTP hydrolysis to physiologically meaningless levels.

Activating Ras mutations occur in 30% of human cancers. Specific RAS genes are mutated in different malignancies: KRAS mutations are prevalent in pancreatic, colorectal, endometrial, biliary tract, lung and cervical cancers; KRAS and NRAS gene mutations are found in myeloid malignancies; and NRAS and HRAS gene mutations predominate in melanoma and bladder cancer, respectively.

Broadly, the present invention is contemplated to be a method of treating all Ras associated cancers, and particularly, Kras associated cancers, by the steps of identifying those cancers that are Ras associated, and then administering an effective anti-cancer agent, according to the invention.

### B. Identifying RAS Associated Cancers

The step of identifying a Ras associated cancer can be conducted by any art-known process. Known Ras mutations can be identified by taking tumor tissue, e.g., obtained by biopsy, and subjecting the tissue DNA to polymerase chain reaction (PCR) or other art-known amplification methods, with appropriate Ras gene specific primers, and identifying known or new mutations in the amplified DNA. Similarly, tumor tissue RNA can be subjected to reverse transcription/polymerase chain reaction (RT/PCR), and identifying either Ras overexpression and/or known or new mutations in the amplified DNA, relative to wild type sequences. Alternatively, mutein Ras protein can be extracted from the tumor tissue sample and identified by art-known chromatographic or antibody binding properties. The following table summarizes the known mutations associated with Ras cancers.

TABLE 1

| Wild-Type | | |
|---|---|---|
| RAS | SEQ ID | |
| Proteins | NO: | Mutations Found in Cancers |
| Kras2a | SEQ ID | G10G G: G → GG in one individual with AML |
| | NO: 1 | G12A: G → A in colorectal cancer |
| | | G12C: G → C in lung carcinoma |
| | | G12D: G → D in pancreatic carcinoma, stomach |
| | | cancer, breast carcinoma and lung carcinoma |
| | | G12R: G → R in lung cancer and bladder cancer |
| | | G12S: G → S in lung carcinoma and stomach |
| | | cancer |
| | | G12V: G → V in lung carcinoma, pancreatic |
| | | carcinoma, colon cancer and stomach cancer |
| | | A59T: A → T in bladder cancer |
| | | Q61H: Q → H in lung carcinoma |
| | | Q61R: Q → R in colorectal cancer |
| | | K117N: K → N in colorectal cancer |
| | | A146T: A → T in colorectal cancer |
| Kras2b | SEQ ID | As above. This is because the Kras2a and Kras2b |
| | NO: 2 | proteins are homologous below residue 150. |
| Hras | SEQ ID | G61L: Q → L in melanoma |
| | NO: 3 | G12V: G → V in bladder carcinoma |
| Nras | SEQ ID | G12C: G→ C in acute myeloid leukaemia |
| | NO: 4 | G12D: G→D in acute lymphoblastic neoplasm |
| | | G12S: G→S in acute lymphoblastic T cell |
| | | leukaemia |
| | | G12V: G→V in Burkitt lymphoma |
| | | G13D: G→D in Hodgkin lymphoma |
| | | G13R: G→R in acute myeloid leukaemia |
| | | G13V: G→V in acute myeloid leukaemia |
| | | Q61H: Q→H in rhabdomysarcoma |
| | | Q61K: Q→K in adult T cell lyphoma-leukaemia |
| | | Q61L: Q→L in hepatocellular carcinoma |
| | | Q61R: Q→R transition cell carcinoma |
| | | A146T: A→Tacute lymphoblastic B cell leukemia |

Illustrative examples of gene mutations resulting in mutein Ras proteins include Gly12Asp(GGT>GAT), Gly12Ala (GGT>GCT), Gly12Val (GGT>GTT), Gly12Ser (GGT>AGT), Gly12Arg (GGT>CGT), Gly12Cys (GGT>TGT) and Gly13Asp (GGC>GAC). For convenience, the cDNA encoding KRAS is shown as SEQ ID NO: 5, the cDNA encoding HRAS is shown as SEQ ID NO: 6, and the cDNA encoding NRAS is shown as SEQ ID NO: 7.

More particularly, art-known tests identify the presence of KRAS oncogenes in transfection assays which identify Ras p21 by its ability to transform NIH 3T3 cells. Lane, et al., Proc. Natl. Acad. Sci. (U.S.A.), 78:5185 (1981); and B. Shilo, and R. A. Weinberg, Nature, 289:607 (1981), both incorporated by reference herein in their entireties.

Another diagnostic method employs oligonucleotide probes to identify single point mutations in genomic DNA. This technique is based on the observation that hybrids between oligonucleotides form a perfect match with genomic sequences, that is, non-mutated genomic sequences are more stable than those that contain a single mismatch. The latter being a point mutation in p21 associated with the RAS oncogenes. A further diagnostic method is based on the unusual electrophoretic migration of DNA heteroduplexes containing single base mismatches in denaturing gradient gels. Myers et al., Nature, 313:495 (1985), incorporated by reference herein. Refinements of this technique are described by Winter, et al., Proc. Natl. Acad. Sci. (U.S.A.), 82:7575 (1985); and Myers, et al., Science, 230:1242 (1985), both incorporated by reference herein in their entireties.

Further still, antibodies, either polyclonal or monoclonal, have been generated against the intact Ras oncogene p21, or against chemically synthesized peptides having sequences similar to oncogene p21, or the non-transforming counterpart. See, for example, U.S. Pat. No. 4,877,867; EP Patent Publication 108,564 to Cline et al.; Tamura, et al., Cell, 34:587 (1983); PCT Publication No. WO/84/01389 to Weinberg et al.

A number of tests for detecting abnormal RAS genes or mutein Ras proteins in cancer tissue are commercially available. These include, for example, the PCR-based DₓS KRAS Mutation Test Kit, which can be employed for the detection of seven KRAS gene mutations (D_{X}S Diagnostic Innovations, Product Codes KR-03 and KR-04; Manchester, UK). There is also the assay service offered by Targeted Molecular Diagnostics (Westmont, Ill.), marketed as a Real-Time PCR based SNP KRAS assay. Both of these commercially available assays identify common mutations found in the Gly12 and Gly13 hotspots, as follows: Gly12Asp (GGT>GAT), Gly12Ala (GGT>GCT), Gly12Val (GGT>GTT), Gly12Ser (GGT>AGT), Gly12Arg (GGT>CGT), Gly12Cys (GGT>TGT) and Gly13Asp (GGC>GAC)

### C. Compound of Formula (I): Multi-Arm Polymeric Conjugates of 7-Ethyl-10-Hydroxycamptothecin

1. Multi-Arm Polymers

The polymeric prodrugs of 7-ethyl-10-hydroxycamptothecin include four-arm PEG attached to 20-OH group of 7-ethyl-10-hydroxycamptothecin through a bifunctional linker. In one aspect of the present invention, the polymeric prodrugs of 7-ethyl-10-hydroxy-camptothecin include four-arm PEG, prior to conjugation, having the following structure of

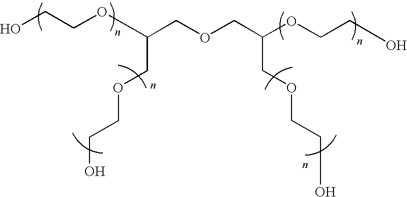

wherein n is a positive integer.
The polymers are those described in NOF Corp. Drug Delivery System catalog, Ver. 8, April 2006, the disclosure of which is incorporated herein by reference.

In one preferred embodiment of the invention, the degree of polymerization for the polymer (n) is from about 28 to about 341 to provide polymers having a total number average molecular weight of from about 5,000 Da to about 60,000 Da, and preferably from about 114 to about 239 to provide polymers having a total number average molecular weight of from 20,000 Da to 42,000 Da. (n) represents the number of repeating units in the polymer chain and is dependent on the molecular weight of the polymer. In one particularly preferred embodiment of the invention, (n) is about 227 to provide the polymeric portion having a total number average molecular weight of about 40,000 Da.

2. Bifunctional Linkers

In certain aspects of the present invention, L is a residue of an amino acid. The amino acid can be selected from any of the known naturally-occurring L-amino acids, e.g., alanine, valine, leucine, isoleucine, glycine, serine, threonine, methionine, cysteine, phenylalanine, tyrosine, tryptophan, aspartic acid, glutamic acid, lysine, arginine, histidine, proline, and/or a combination thereof, to name but a few. In alternative aspects, L can be a peptide residue. The peptide can range in size, for instance, from about 2 to about 10 amino acid residues (e.g., 2, 3, 4, 5, or 6).

Derivatives and analogs of the naturally occurring amino acids, as well as various art-known non-naturally occurring amino acids (D or L), hydrophobic or non-hydrophobic, are also contemplated. Simply by way of example, amino acid analogs and derivates include: 2-aminoadipic acid, 3-aminoadipic acid, beta-alanine, beta-aminopropionic acid, 2-aminobutyric acid, 4-aminobutyric acid, piperidinic acid, 6-aminocaproic acid, 2-aminoheptanoic acid, 2-aminoisobutyric acid, 3-aminoisobutyric acid, 2-aminopimelic acid, 2,4-aminobutyric acid, desmosine, 2,2-diaminopimelic acid, 2,3-diaminopropionic acid, N-ethylglycine, N-ethylasparagine, 3-hydroxyproline, 4-hydroxyproline, isodesmosine, allo-isoleucine, N-methylglycine or sarcosine, N-methyl-isoleucine, N-methyllysine, N-methylvaline, norvaline, norleucine, ornithine, and others too numerous to mention, that are listed in 63 Fed. Reg., 29620, 29622, incorporated by reference herein. Some preferred L groups include glycine, alanine, methionine or sarcosine residues. For example, the compounds can be among:

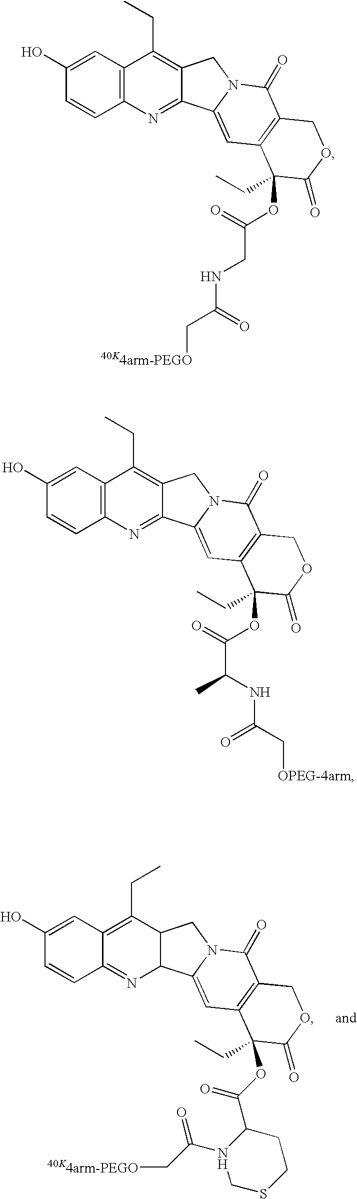

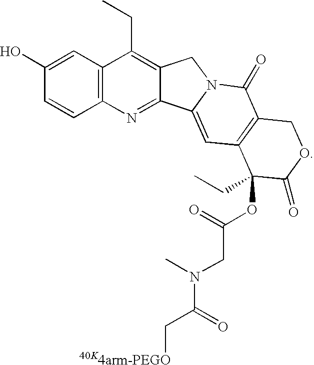

For ease of the description and not limitation, one arm of the four-arm PEG is shown. One arm, up to four arms of the four-arm PEG can be conjugated with 7-ethyl-10-hydroxy-camptothecin.

More preferably, compounds of the present invention include a glycine residue as the linker group (L).

Alternatively, L after attachment between the polymer and 7-ethyl-10-hydroxy-camptothecin is selected among:

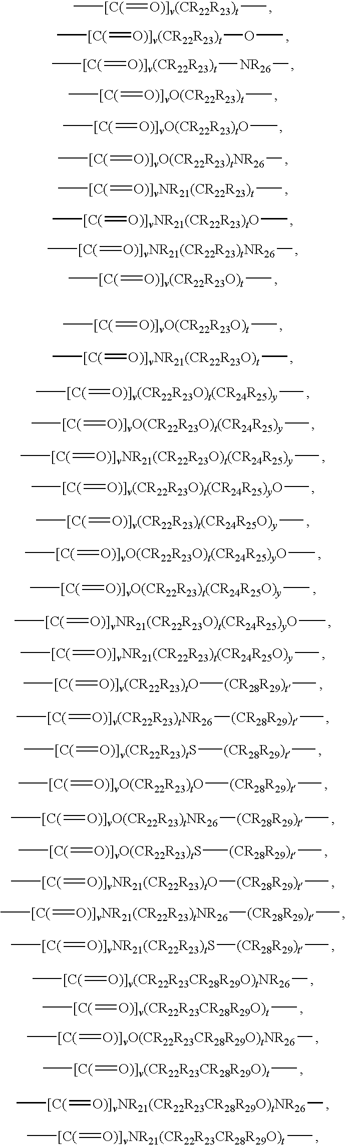

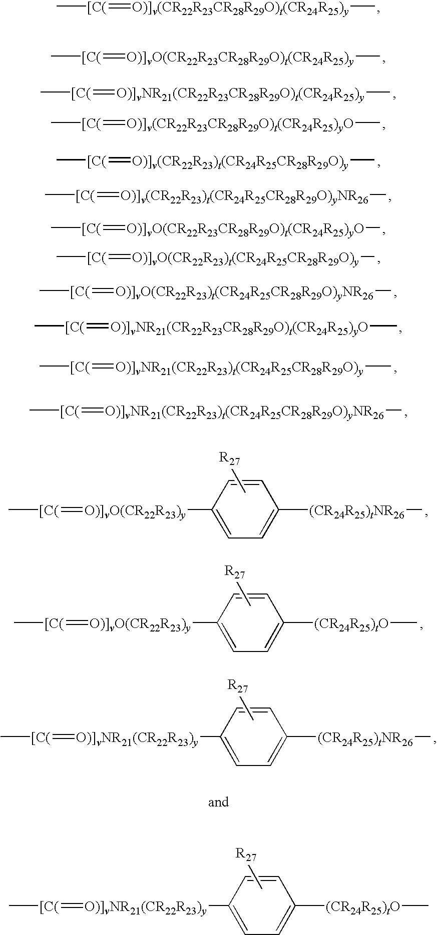

wherein:

R₂₁-R₂₉ are independently selected among hydrogen, amino, substituted amino, azido, carboxy, cyano, halo, hydroxyl, nitro, silyl ether, sulfonyl, mercapto, C₁₋₆ alkylmercapto, arylmercapto, substituted arylmercapto, substituted C₁₋₆ alkylthio, C₁₋₆ alkyls, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆heteroalkyl, C₁₋₆ alkoxy, aryloxy, C₁₋₆ heteroalkoxy, heteroaryloxy, 2-6 alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy, substituted and arylcarbonyloxy;

(t), (t′) and (y) are independently chosen from zero or a positive integer, preferably from about 1 to about 10 such as 1, 2, 3, 4, 5 and 6; and

(v) is 0 or 1.

In some preferred embodiments, L can include:

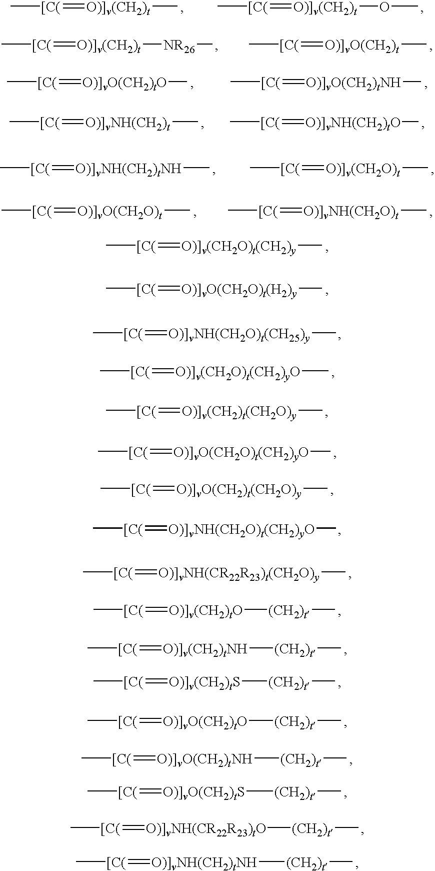

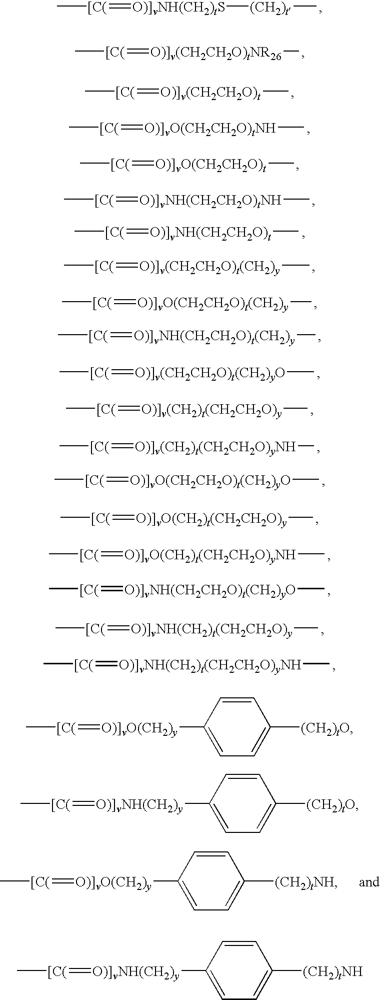

wherein (t), (t′) and (y) are independently chosen from zero or a positive integer, preferably from about 1 to about 10 (e.g., 1, 2, 3, 4, 5, and 6); and

(v) is 0 or 1.

In some aspects of the present invention, the compounds include from 1 to about 10 units (e.g., 1, 2, 3, 4, 5, or 6) of the bifunctional linker. In some preferred aspects of the present invention, the compounds include one unit of the bifunctional linker and thus (m) is 1.

Additional linkers are found in Table 1 of Greenwald et al. (Bioorganic & Medicinal Chemistry, 1998, 6:551-562), the contents of which are incorporated by reference herein.

3. Alternative Polymers

In an alternative embodiment, the inventive method is conducted by administering polymeric conjugate of 7-ethyl-10-hydroxycamptothecin to a mammal having a Ras associated cancer. The polymeric conjugate includes a compound of Formula (II) or (III):

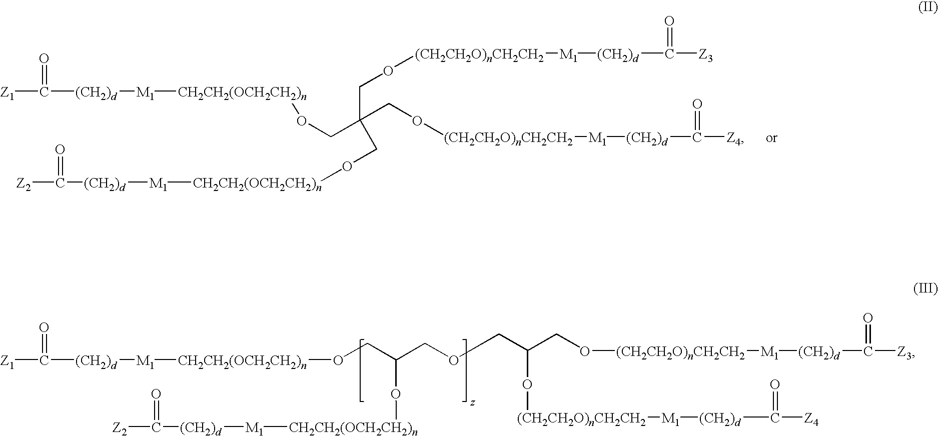

wherein

Z₁, Z₂, Z₃ and Z₄ are independently OH or (L)ₘ-D;

L is a bifunctional linker;

D is 7-ethyl-10-hydroxycamptothecin;

M₁ is O, S, or NH;

(d) is zero or a positive integer of from about 1 to about 10, preferably, 0, 1, 2, or 3, and more preferably, 0 or 1;

(z) is zero or a positive integer of from 1 to about 29, preferably, 1, 5, 13 or 29;

(m) is 0 or a positive integer, preferably zero or an integer from about 1 to about 10

(e.g., 1, 2, 3, 4, 5, 6), wherein each L is the same or different when (m) is equal to or greater than 2; and

(n) is a positive integer of from about 10 to about 2,300 so that the polymeric portion of the compound has the total number average molecular weight of from about 2,000 to about 100,000 daltons, provided that Z₁, Z₂, Z₃ and Z₄ are not all OH.

The 7-ethyl-10-hydroxycamptothecin can be a racemic mixture or an optically pure isomer. Preferably, a substantially pure and active form of such as the 20(S) camptothecin or camptothecin analog is employed in the multi-arm polymeric prodrugs.

### D. Synthesis of Prodrugs

Generally, the polymeric 7-ethyl-10-hydroxycamptothecin prodrugs described herein are prepared by reacting one or more equivalents of an activated multi-arm polymer with, for example, one or more equivalents per active site of amino acid-(20)-7-ethyl-10-hydroxycamptothecin compound under conditions which are sufficient to effectively cause the amino group to undergo a reaction with the carboxylic acid of the polymer and form a linkage. Details of the synthesis are described in U.S. Pat. No. 7,462,627, entitled “Multi-arm Polymeric Conjugates of 7-Ethyl-10-hydroxycamptothecin For Treatment of Breast Colorectal, Pancreatic, Ovarian and Lung Cancers”, the contents of which are incorporated herein by reference in its entirety.

More specifically, the methods can include:

1) providing one equivalent of 7-Ethyl-10-hydroxycamptothecin containing an available 20-hydroxyl group and one or more equivalents of a bifunctional linker containing an available carboxylic acid group;

2) reacting the two reactants to form a camptothecin-bifunctional linker intermediate in an inert solvent such as dichloromethane (“DCM”) (or dimethylformamide (“DMF,” chloroform, toluene or mixtures thereof) in the presence of a coupling reagent such as 1,(3-dimethyl aminopropyl) 3-ethyl carbodiimide (EDC), (or 1,3-diisopropylcarbodiimide (DIPC), any suitable dialkyl carbodiimide, Mukaiyama reagents, (e.g. 2-halo-1-alkyl-pyridinium halides) or propane phosphonic acid cyclic anhydride (PPACA), etc) and a suitable base such as N,N-dimethylpyridin-4-amine (“DMAP”); and

3) reacting one or more equivalents per active site (2 eq. in Example) of the resulting intermediate having an amine group and one equivalent of an activated polymer, such as a PEG-acid in an inert solvent such as DCM (or DMF, chloroform, toluene or mixtures thereof) in the presence of a coupling reagent such as 1,(3-dimethyl aminopropyl) 3-ethyl carbodiimide (EDC), PPAC (or 1,3-diisopropylcarbodiimide (DIPC), any suitable dialkyl carbodiimide, Mukaiyama reagents, (e.g. 2-halo-1-alkyl-pyridinium halides) or propane phosphonic acid cyclic anhydride (PPACA), etc.), and a suitable base such as DMAP, which are available, for example, from commercial sources such as Sigma Chemical, or synthesized using known techniques, at a temperature from 0° C. up to 22° C.

In one preferred aspect, the 10-hydroxyl group of 7-ethyl-10-hydroxycamptothecin is protected prior to step 1).

Protection of the aromatic OH of camptothecin analogs, such as the 10-hydroxyl group in 7-ethyl-10-hydroxycamptothecin, is preferred because the protected 7-ethyl-10-hydroxycamptothecin intermediates thereof have better solubility and can be purified in highly pure form efficiently and effectively. For example, silyl-containing protecting groups such as TBDPSCl (t-butyldiphenylsilyl chloride), TBDMSCl (t-butyldimethylsilyl chloride) and TMSCl (trimethylsilyl chloride) can be used to protect the 10-hydroxyl group in 7-ethyl-10-hydroxycamptothecin.

The activated polymer, i.e., a polymer containing 1-4 terminal carboxyl acid groups can be prepared, for example, by converting NOF Sunbright-type having terminal OH groups into the corresponding carboxyl acid derivatives using standard techniques well known to those of ordinary skill. See, for example, Examples 1-2 herein, as well as commonly assigned U.S. Pat. No. 5,605,976 and U.S. Patent Publication No. 2007/0173615, the contents of each of which are incorporated herein by reference.

The first and second coupling agents can be the same or different.

Examples of preferred bifunctional linker groups include glycine, alanine, methionine, sarcosine, etc. and syntheses are shown in the Examples. Alternative and specific syntheses are provided in the examples.

For example, the compounds of the present invention prepared according to the processes are among:

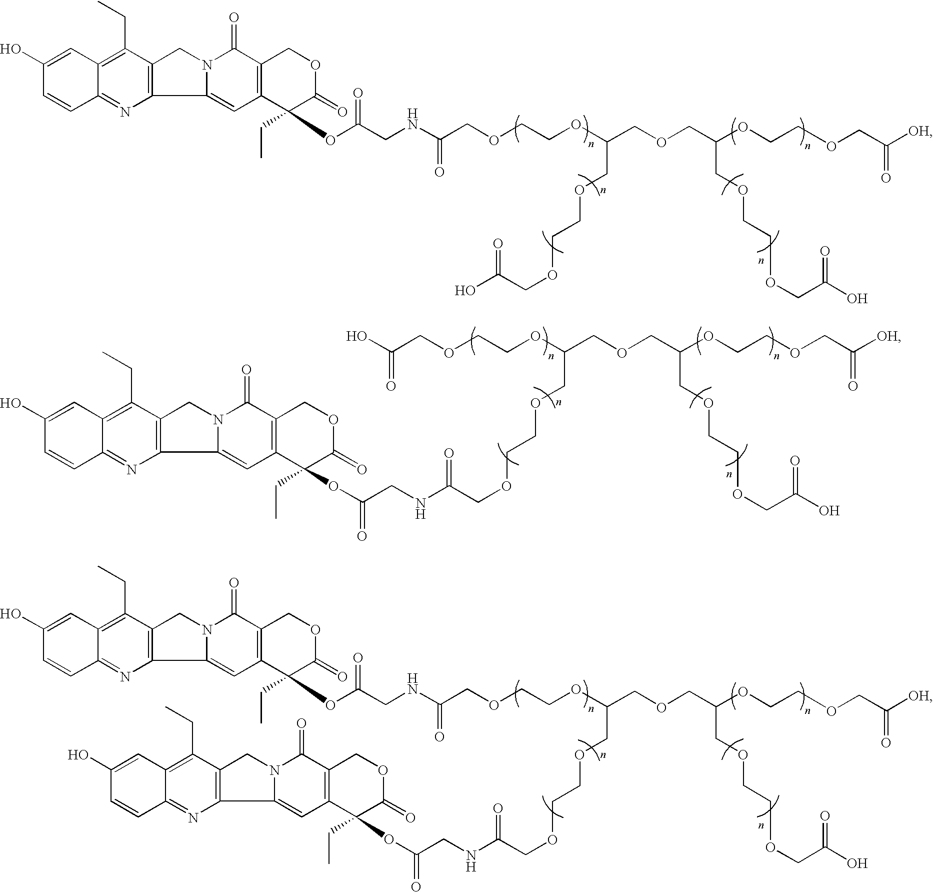

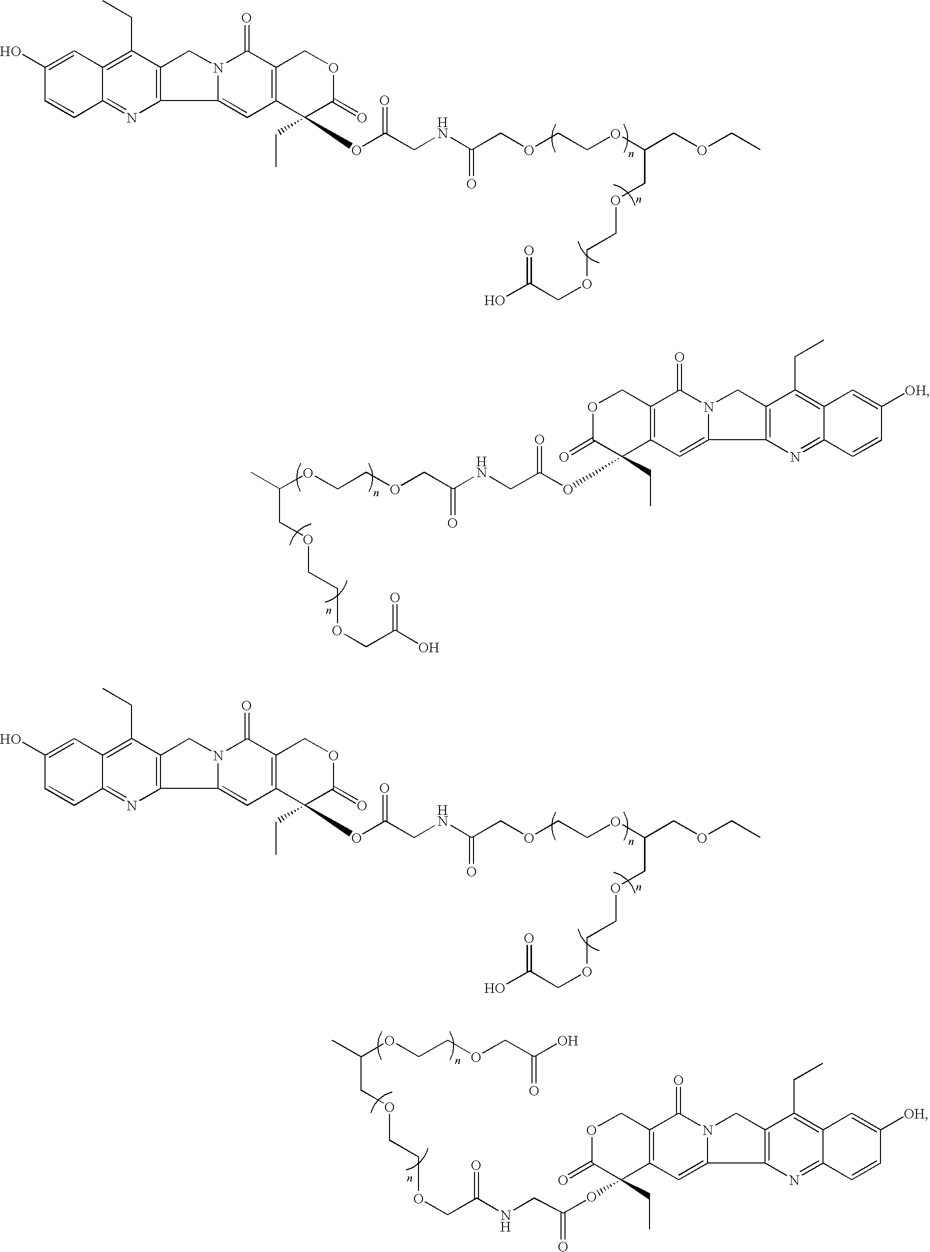

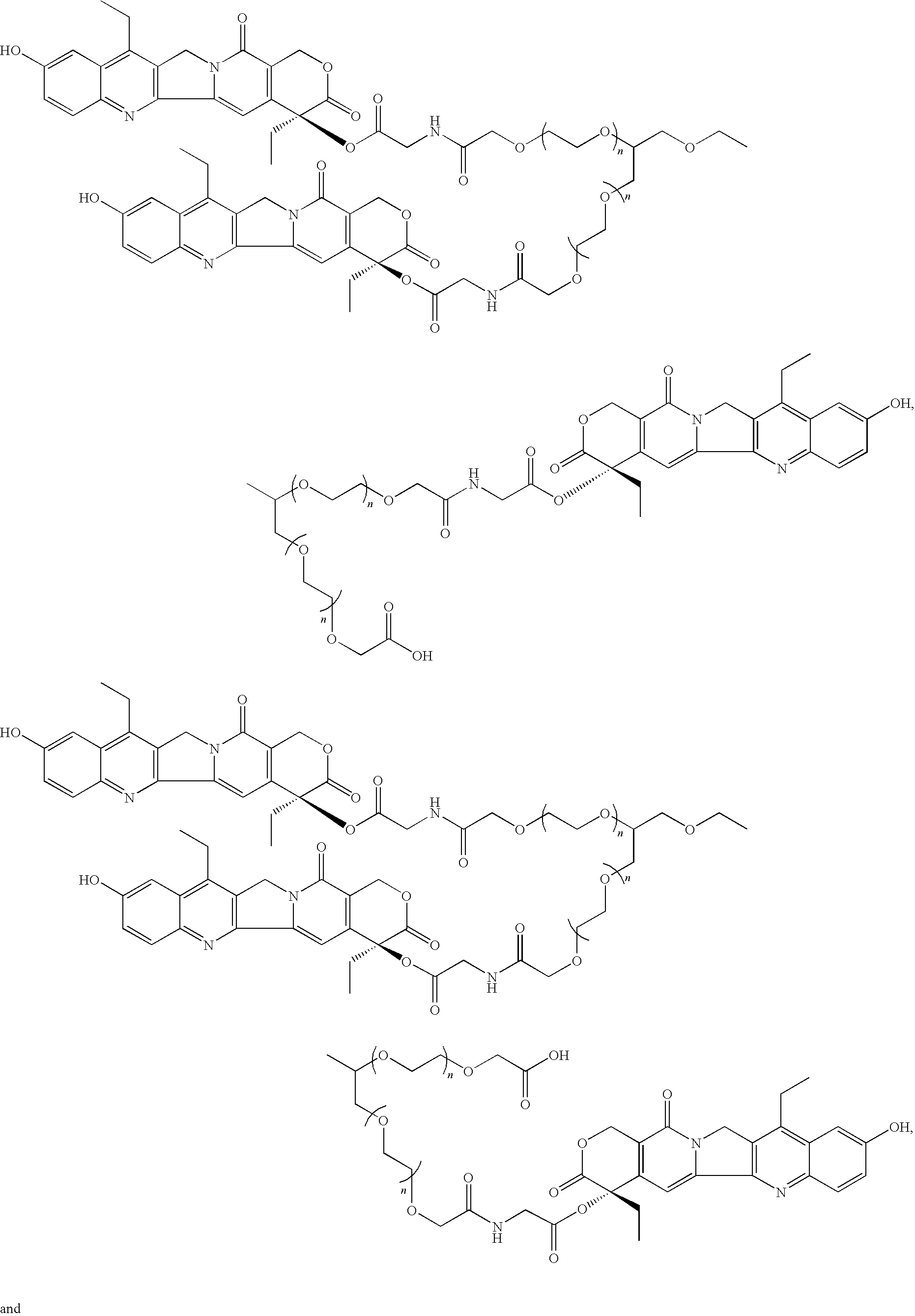

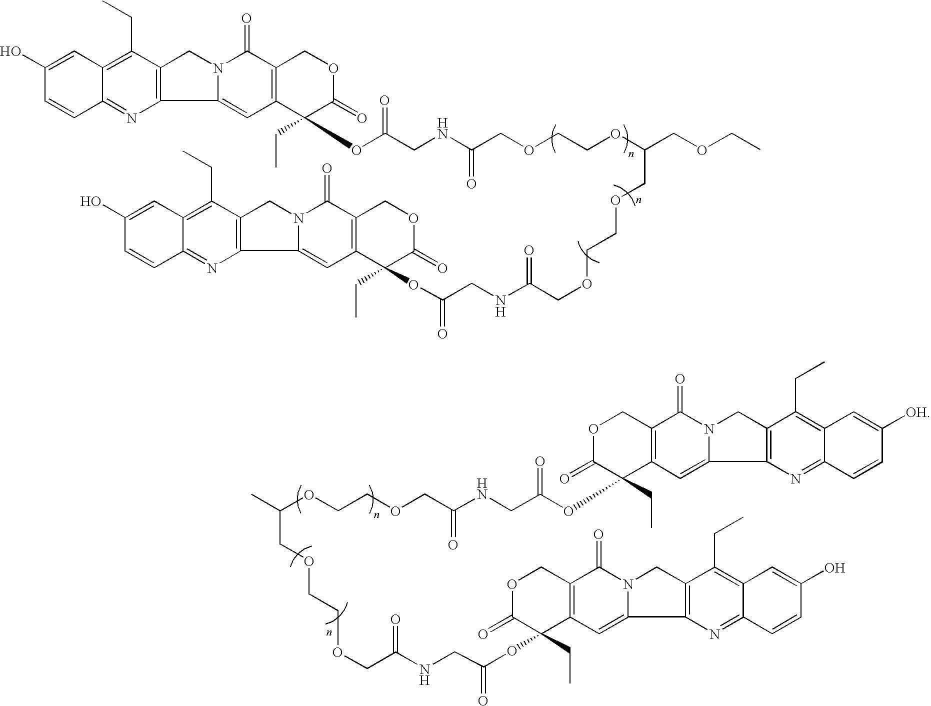

One particularly preferred compound is

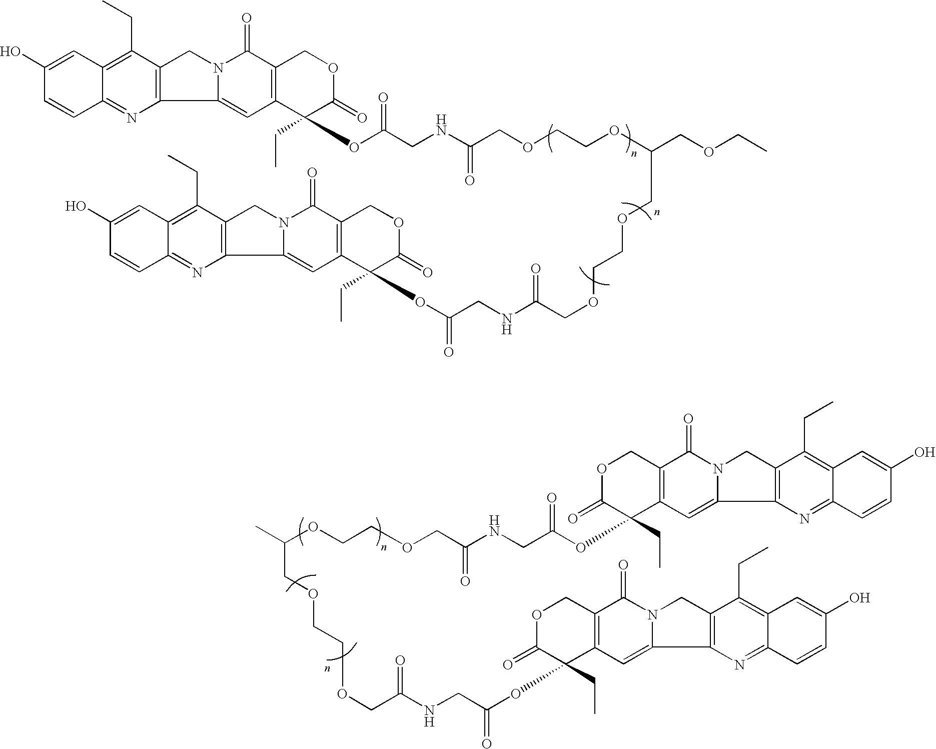

wherein all four arms of the polymer are conjugated to 7-ethyl-10-hydroxycamptothecin through glycine. HPLC analysis of compounds made in accordance with this aspect of the inventions shows that on average, four 7-ethyl-10-hydroxycamptothecin molecules are conjugated to one PEG molecule (4% by weight).

### E. Compositions/Formulations

Pharmaceutical compositions containing the polymer conjugates of the present invention may be manufactured by processes well known in the art, e.g., using a variety of well-known mixing, dissolving, granulating, levigating, emulsifying, encapsulating, entrapping or lyophilizing processes. The compositions may be formulated in conjunction with one or more physiologically acceptable carriers comprising excipients and auxiliaries which facilitate processing of the active compounds into preparations which can be used pharmaceutically. Proper formulation is dependent upon the route of administration chosen. Parenteral routes are preferred in many aspects of the invention.

For injection, including, without limitation, intravenous, intramuscular and subcutaneous injection, the compounds of the invention may be formulated in aqueous solutions, preferably in physiologically compatible buffers such as physiological saline buffer or polar solvents including, without limitation, a pyrrolidone or dimethylsulfoxide.

The compounds may also be formulated for parenteral administration, e.g., by bolus injection or continuous infusion. Formulations for injection may be presented in unit dosage form, e.g., in ampoules or in multi-dose containers. Useful compositions include, without limitation, suspensions, solutions or emulsions in oily or aqueous vehicles, and may contain adjuncts such as suspending, stabilizing and/or dispersing agents. Pharmaceutical compositions for parenteral administration include aqueous solutions of a water soluble form, such as, without limitation, a salt (preferred) of the active compound. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances that increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions. Alternatively, the active ingredient may be in powder form for constitution with a suitable vehicle, e.g., sterile, pyrogen-free water, before use.

For oral administration, the compounds can be formulated by combining the active compounds with pharmaceutically acceptable carriers well-known in the art. Such carriers enable the compounds of the invention to be formulated as tablets, pills, lozenges, dragees, capsules, liquids, gels, syrups, pastes, slurries, solutions, suspensions, concentrated solutions and suspensions for diluting in the drinking water of a patient, premixes for dilution in the feed of a patient, and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars, including lactose, sucrose, mannitol, or sorbitol, cellulose preparations such as, for example, maize starch, wheat starch, rice starch and potato starch and other materials such as gelatin, gum tragacanth, methyl cellulose, hydroxypropyl-methylcellulose, sodium carboxy-methylcellulose, and/or polyvinylpyrrolidone (PVP). If desired, disintegrating agents may be added, such as cross-linked polyvinyl pyrrolidone, agar, or alginic acid. A salt such as sodium alginate may also be used.

For administration by inhalation, the compounds of the present invention can conveniently be delivered in the form of an aerosol spray using a pressurized pack or a nebulizer and a suitable propellant.

The compounds may also be formulated in rectal compositions such as suppositories or retention enemas, using, e.g., conventional suppository bases such as cocoa butter or other glycerides.

In addition to the formulations described previously, the compounds may also be formulated as depot preparations. Such long acting formulations may be administered by implantation (for example, subcutaneously or intramuscularly) or by intramuscular injection. A compound of this invention may be formulated for this route of administration with suitable polymeric or hydrophobic materials (for instance, in an emulsion with a pharmacologically acceptable oil), with ion exchange resins, or as a sparingly soluble derivative such as, without limitation, a sparingly soluble salt.

Other delivery systems such as liposomes and emulsions can also be used.

Additionally, the compounds may be delivered using a sustained-release system, such as semi-permeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the particular compound, additional stabilization strategies may be employed.

### G. Dosages

Determination of a therapeutically effective amount is well within the capability of those skilled in the art, especially in light of the disclosure herein. For any compound used in the methods of the invention, the therapeutically effective amount can be estimated initially from in vitro assays. Then, the dosage can be formulated for use in animal models so as to achieve a circulating concentration range that includes the effective dosage. Such information can then be used to more accurately determine dosages useful in patients.

The compositions may be administered once daily or divided into multiple doses which can be given as part of a multi-week treatment protocol. The precise dose will depend on the stage and severity of the condition, the susceptibility of the tumor to the polymer-prodrug composition, and the individual characteristics of the patient being treated, as will be appreciated by one of ordinary skill in the art.

The amount of the composition, e.g., used as a prodrug, that is administered will depend upon the parent molecule included therein (in this case, 7-ethyl-10-hyroxycamptothecin). Generally, the amount of prodrug used in the treatment methods is that amount which effectively achieves the desired therapeutic result in mammals. Naturally, the dosages of the various prodrug compounds can vary somewhat depending upon the parent compound, rate of in vivo hydrolysis, molecular weight of the polymer, etc. In addition, the dosage, of course, can vary depending upon the dosage form and route of administration.

In general, however, the polymeric ester derivatives of 7-ethyl-10-hyroxycamptothecin described herein can be administered in amounts ranging from about 0.3 to about 90 mg/m² and preferably about 0.6 to about 45 mg/m²/dose, yet preferably from about 1 to about 18 mg/m²/dose for systemic delivery. The compounds can be administered in amounts ranging from about 0.3 to about 90 mg/m²/week such as, for example, from about 0.9 to about 18 mg/m² week. In particular embodiments, the dose regimens can be, for example, from 5-7 mg/m² weekly for 3 weeks in 4-week cycles, from 1.25-45 mg/m² one injection every 3 weeks, and/or from 1-16 mg/m² as three injections weekly in a four week cycle.

Alternatively and preferably, the amounts of the compounds described herein range from about 1 to about 18 mg/m² body surface/dose. Some preferred doses include one of the following: 1.25, 2.5, 5, 9, 10, 12, 13, 14, 15, 16 and 16.5 mg/m²/dose. Preferably, the amounts administered can range from about 1.25 to about 16.5 mg/m² body surface/dose. One preferred dosage includes 5 mg/m² body surface/dose.

The polymeric ester derivatives of 7-ethyl-10-hyroxycamptothecin (e.g., according to Formulas (I), (II) or (III) described herein can be administered in amounts ranging from about 0.3 to about 90 mg/m² body surface/week such as, for example, from about 1 to about 18 mg/m² body surface/week. In particular embodiments, the dose regimens can be, for example, from about 5 to about 7 mg/m² body surface weekly for 3 weeks in 4-week cycles, from about 1.25 to about 45 mg/m² one injection every 3 weeks, and/or from about 1 to about 16 mg/m² three injections weekly in a four week cycle.

The treatment protocol can be based on a single dose administered once every three weeks or divided into multiple doses which are given as part of a multi-week treatment protocol. Thus, the treatment regimens can include one dose every three weeks for each treatment cycle and, alternatively one dose weekly for three weeks followed by one week off for each cycle.

The weight given above represent the weight of 7-ethyl-10-hydroxycamptothecin present in the PEG-conjugated 7-ethyl-10-hydroxycamptothecin employed for treatment. The actual weight of the PEG-conjugated 7-ethyl-10-hydroxycamptothecin will vary depending on the loading of the PEG (e.g., optionally from one to four moles of 7-ethyl-10-hydroxycamptothecin per mole of PEG.

The range set forth above is illustrative and those skilled in the art will determine the optimal dosing of the prodrug selected based on clinical experience and the treatment indication. Moreover, the exact formulation, route of administration and dosage can be selected by the individual physician in view of the patient's condition. Additionally, toxicity and therapeutic efficacy of the compounds described herein can be determined by standard pharmaceutical procedures in cell cultures or experimental animals using methods well-known in the art.

The precise dose will depend on the stage and severity of the condition, and the individual characteristics of the patient being treated, as will be appreciated by one of ordinary skill in the art. It is also contemplated that the treatment continues until satisfactory results are observed, which can be as soon as after 1 cycle although from about 3 to about 6 cycles or more cycles may be required.

In some preferred embodiments, the treatment protocol includes administering the amount ranging from about 1.25 to about 16.5 mg/m² body surface/dose weekly for three weeks, followed by one week without treatment and repeating for about 3 cycles or more until the desired results are observed. Alternatively, the compounds described herein can be administered in the amount ranging from about 1.25 to about 16.5 mg/m² body surface/dose every three weeks repeating for about 3 cycles or more. The amount administered per each cycle can range more preferably from about 2.5 to about 16.5 mg/m² body surface/dose.

In one particular embodiment, the polymeric ester derivatives of 7-ethyl-10-hyroxycamptothecin can be administered one dose such as 5, 9 or 10 mg/m² weekly for three weeks, followed by one week without treatment in treatment of lung, colon or pancreatic cancer. The dosage of treatment cycle can be designed as an escalating dose regimen when two or more treatment cycles are applied. The polymeric drug is preferably administered via IV infusion.

In another particular embodiment, the compound of Formula (I) (or Formula (II) or (III)) is administered in a dose from about 1 to about 16 mg/m² body surface/dose. The dose can be given weekly. The treatment protocol includes administering the compound of Formula I, Formula II or III) in amounts ranging from about 12 to about 16 mg/m² body surface/dose weekly for three weeks, followed by one week without treatment.

In yet another particular embodiment, the dose regiment can be about 10 mg/m² body surface/dose every three weeks.

Alternative embodiments include: for the treatment of pediatric patients, a regimen based on a protocol of about 1.85 mg/m² body surface/dose daily for 5 days every three weeks, a protocol of from about 1.85 to about 7.5 mg/m² body surface/dose daily for 3 days every 25 days, or a protocol of about 22.5 mg/m² body surface/dose once every three weeks, and for the treatment of adult patients, a protocol based on about 13 mg/m² body surface/dose every three weeks or about 4-5 mg/m² body surface/dose weekly for four weeks every six weeks. The compounds described herein can be administered in combination with a second therapeutic agent. In one embodiment, the combination therapy includes a protocol of about 0.75 mg/m² body surface/dose daily for 5 days each cycle in combination with a second agent.

Alternatively, the compounds can be administered based on body weight. The dosage range for systemic delivery of a compound of Formula (I) (or Formula (II) or (III)) in a mammal will be from about 1 to about 100 mg/kg/week and is preferably from about 2 to about 60 mg/kg/week. Thus, the amounts can range from about 0.1 mg/kg body weight/dose to about 30 mg/kg body weight/dose, preferably, from about 0.3 mg/kg to about 10 mg/kg. Specific doses such as 5 or 10 mg/kg at q2d×5 regimen (multiple dose) or 20 or 30 mg/kg on a single dose regimen can be administered.

In all aspects of the invention where polymeric conjugates are administered, the dosage amount mentioned is based on the amount of 7-ethyl-10-hydroxycamptothecin rather than the amount of polymeric conjugate administered. It is contemplated that the treatment will be given for one or more cycles until the desired clinical result is obtained. The exact amount, frequency and period of administration of the compound of the present invention will vary, of course, depending upon the sex, age and medical condition of the patient as well as the severity of the disease as determined by the attending clinician.

Still further aspects include combining the therapy described herein with other anticancer therapies for synergistic or additive benefit.

### H. Methods of Treatment

In view of the above, there are also provided methods of treating Ras associated cancer in a mammal, comprising administering an effective amount of multi-arm polymeric prodrugs of 7-ethyl-10-hydroxycamptothecin, e.g., four arm PEGylated 7-ethyl-10-hydroxycamptothecin, to a patient determined as having a Ras associated cancer.

The compositions are useful for, among other things, treating neoplastic disease, reducing tumor burden, preventing metastasis of Ras associated neoplasms and preventing recurrences of Ras associated tumor/neoplastic growths in mammals. In alternative aspects, the Ras associated cancer being treated can be one or more of the following: solid tumors, colorectal cancer, pancreatic cancer, lung cancer, small cell lung cancer, stomach cancer, lymphomas, acute lymphocytic leukemia (ALL), melanoma, acute myeloid leukemia (AML), breast cancer, bladder cancer, glioblastoma, ovarian cancer, non-Hodgkin's lymphoma, anal cancer, head and neck cancer. In one embodiment, the methods include administering the compounds described herein to a mammal having a Ras associated cancer such as lung cancer, colorectal cancer and pancreatic cancer.

In another aspect of the invention, the methods of treatment include administering an effective amount of the compounds described herein to a mammal or patient having a Ras associated cancer and a resistant or refractory cancer to camptothecin, CPT-11, an epidermal growth factor receptor antagonist (for example, Erbitux® cetuximab or C225) therapies and combinations thereof.

### EXAMPLES

The following examples serve to provide farther appreciation of the invention but are not meant in any way to restrict the effective scope of the invention. The bold-faced numbers, e.g., compound numbers, recited in the Examples correspond to those shown in the figures.

General Procedures. All reactions were run under an atmosphere of dry nitrogen or argon. Commercial reagents were used without further purification. All PEG compounds were dried under vacuum or by azeotropic distillation from toluene prior to use. ¹³C NMR spectra were obtained at 75.46 MHz using a Varian Mercury® 300 NMR spectrometer and deuterated chloroform and methanol as the solvents unless otherwise specified. Chemical shifts (δ) are reported in parts per million (ppm) downfield from tetramethylsilane (TMS).
HPLC Method. The reaction mixtures and the purity of intermediates and final products were monitored by a Beckman Coulter System Gold® HPLC instrument. It employs a ZOBAX® 300SB C8 reversed phase column (150×4.6 mm) or a Phenomenex Jupiter® 300A C18 reversed phase column (150×4.6 mm) with a multiwavelengh UV detector, using a gradient of 10-90% of acetonitrile in 0.05% trifluoroacetic acid (TFA) at a flow rate of 1 mL/min.)

### Example 1

### ^{40k}4arm-PEG-tBu ester (compound 2)

^{40k}4arm-PEG-OH (12.5 g, 1 eq.) was azeotroped with 220 mL of toluene to remove 35 mL of toluene/water. The solution was cooled to 30° C. and 1.0 M potassium t-butoxide in t-butanol (3.75 mL, 3 eq×4=12 eq.) was added. The mixture was stirred at 30° C. for 30 min and then t-butyl bromoacetate (0.975 g, 4 eq.×4=16 eq.) was added. The reaction was kept at 30° C. for 1 hour and then was cooled to 25° C. 150 mL of ether was slowly added to precipitate product. The resulting suspension was cooled to 17° C. and stayed at 17° C. for half hour. The crude product was filtered and the wet cake was washed with ether twice (2×125 mL). The isolated wet cake was dissolved in 50 ml of DCM and the product was precipitated with 350 ml of ether and filtered. The wet cake was washed with ether twice (2×125 mL). The product was dried under vacuum at 40° C. (yield=98%, 12.25 g). ¹³C NMR (75.4 MHz, CDCl₃): δ 27.71, 68.48-70.71 (PEG), 80.94, 168.97.

### Example 2

### ^{40k}4arm-PEG Acid (compound 3)

^{40k}4arm-PEG-tBu ester (compound 2, 12 g) was dissolved in 120 mL of DCM and then 60 mL of TEA were added. The mixture was stirred at room temperature for 3 hours and then the solvent was removed under vacuum at 35° C. The resulting oil residue was dissolved in 37.5 mL of DCM. The crude product was precipitated with 375 mL of ether. The wet cake was dissolved in 30 mL of 0.5% NaHCO₃. The product was extracted with DCM twice (2×150 ml). The combined organic layers were dried over 2.5 g of MgSO₄. The solvent was removed under vacuum at room temperature. The resulting residue was dissolved in 37.5 mL of DCM and the product was precipitated with 300 mL of ether and filtered. The wet cake was washed with ether twice (2×125 ml). The product was dried under vacuum at 40° C. (yield=90%, 10.75 g). ¹³C NMR (75.4 MHz, CDCl₃): δ 67.93-71.6 (PEG), 170.83.

### Example 3

### TBDPS-(10)-(7-ethyl-10-hydroxycamptothecin) (compound 5)

To a suspension of 7-ethyl-10-hydroxycamptothecin (compound 4, 2.0 g, 5.10 mmol, 1 eq.) in 100 mL of anhydrous DCM were added Et₃N (4.3 mL, 30.58 mmol, 6 eq.) and TBDPSCl (7.8 mL, 30.58 mmol, 6 eq.). The reaction mixture was heated to reflux overnight and then, was washed with a 0.2 N HCl solution (2×50 mL), a saturated NaHCO₃ solution (100 mL) and brine (100 mL). The organic layer was dried over MgSO₄, filtered and evaporated under vacuum. The residue was dissolved in anhydrous DCM and precipitated by addition of hexanes. The precipitation with DCM/hexanes was repeated to get rid of excess TBDPSCl. The solids were filtered and dried under vacuum to give 2.09 g of product. (65% yield). ¹H NMR (300 MHz, CDCl₃): δ 0.90 (3H, t, J=7.6 Hz), 1.01 (3H, t, J=7.3 Hz), 1.17 (9H, s), 1.83-1.92 (2H, m), 2.64 (2H, q, 6.9 Hz), 3.89 (1H, s, OH), 5.11 (2H, s), 5.27 (1H, d, J=16.1 Hz), 5.72 (1H, d, J=16.4 Hz), 7.07 (2H, d, J=2.63 Hz), 7.36-7.49 (7H, m), 7.58 (1H, s), 7.75-7.79 (4H, m), 8.05 (1H, d, J=9.4 Hz). ¹³C NMR (75.4 MHz, CDCl₃): δ 7.82, 13.28, 19.52, 22.86, 26.48, 31.52, 49.23, 66.25, 72.69, 97.25, 110.09, 117.57, 125.67, 126.57, 127.65, 127.81, 130.02, 131.69, 131.97, 135.26, 143.51, 145.05, 147.12, 149.55, 149.92, 154.73, 157.43, 173.72.

### Example 4

### TBDPS-(10)-(7-ethyl-10-hydroxycamptothecin)-(20)-Gly-Boc (compound 6)

To a 0° C. solution of TBDPS-(10)-(7-ethyl-10-hydroxycamptothecin) (compound 5, 3.78 g, 5.99 mmol, 1 eq.) and Boc-Gly-OH (1.57 g, 8.99 mmol, 1.5 eq.) in 100 mL of anhydrous DCM was added EDC (1.72 g, 8.99 mmol, 1.5 eq.) and DMAP (329 mg, 2.69 mmol, 0.45 eq.). The reaction mixture was stirred at 0° C. until HPLC showed complete disappearance of the starting material (approx. 1 hour and 45 minutes). The organic layer was washed with a 0.5% NaHCO₃ solution (2×50 mL), water (1×50 mL), a 0.1 N HCl solution (2×50 mL) and brine (1×50 mL); and dried over MgSO₄. After filtration and evaporation under vacuum, 4.94 g of crude product were obtained (quantitative yield). The crude solid was used in the next reaction without further purification. ¹H NMR (300 MHz, CDCl₃): δ 0.89 (3H, t, J=7.6 Hz), 0.96 (3H, t, J=7.5 Hz), 1.18 (9H, s), 1.40 (9H, s), 2.07-2.29 (3H, m), 2.64 (2H, q, 7.5 Hz), 4.01-4.22 (2H, m), 5.00 (1H, br s), 5.01 (2H, s), 5.37 (1H, d, J=17.0 Hz), 5.66 (1H, d, J=17.0 Hz), 7.08 (1H, d, J=2.34 Hz), 7.16 (1H, s), 7.37-7.50 (7H, m), 7.77 (4H, d, J=7.6 Hz), 8.05 (1H, d, J=9.4 Hz). ¹³C NMR (75.4 MHz, CDCl₃): δ 7.52, 13.30, 19.50, 22.86, 26.45, 28.21, 31.64, 42.28, 49.14, 67.00, 76.65, 79.96, 95.31, 110.13, 118.98, 125.75, 126.45, 127.68, 127.81, 130.03, 131.54, 131.92, 135.25, 143.65, 144.91, 145.19, 147.08, 149.27, 154.75, 155.14, 157.10, 166.98, 169.17.

### Example 5

### TBDPS-(10)-(7-ethyl-10-hydroxycamptothecin)-(20)-Gly•HCl (compound 7)

To a solution of TBDPS-(10)-(7-ethyl-10-hydroxycamptothecin)-(20)-Gly-Boc (compound 6, 1 g, 1.27 mmol) in 5 mL anhydrous dioxane was added 5 mL of a 4 M solution of HCl in dioxane. The reaction mixture was stirred at room temperature until HPLC showed complete disappearance of the starting material (1 hour). The reaction mixture was added to 50 mL of ethyl ether and the resulting solid was filtered. The solid was dissolved in 50 mL DCM and washed with brine (pH was adjusted to 2.5 by addition of a saturated NaHCO₃ solution). The organic layer was dried over MgSO₄, filtered and evaporated under vacuum. The residue was dissolved in 5 mL of DCM and precipitated by addition of 50 mL ethyl ether. Filtration afforded 770 mg (84% yield) final product. ¹H NMR (300 MHz, CDCl₃): δ 0.84 (3H, t, J=7.6 Hz), 1.05 (3H, t, J=7.3 Hz), 1.16 (9H, s), 2.15-2.30 (3H, m), 2.59 (2H, q, 7.6 Hz), 4.16 (1H, d, J=17.9 Hz), 4.26 (1H, d, J=17.9 Hz), 5.13 (2H, s), 5.46 (1H, d, J=17.0 Hz), 5.60 (1H, d, J=17.0 Hz), 7.11 (1H, d, J=2.34 Hz), 7.30 (1H, s), 7.40-7.51 (6H, m), 7.56 (1H, dd, J=2.34, 9.4 Hz), 7.77 (4H, dd, J=7.6, 1.6 Hz), 7.98 (1H, d, J=9.1 Hz). ¹³C NMR (75.4 MHz, CDCl₃): δ 8.09, 13.72, 20.26, 23.61, 26.94, 31.83, 41.01, 50.71, 67.62, 79.51, 97.03, 111.65, 119.69, 127.13, 128.97, 128.99, 129.11, 131.43, 131.96, 133.00, 133.03, 136.51, 145.62, 145.81, 147.24, 148.29, 150.58, 156.27, 158.68, 167.81, 168.34.

### Example 6

### ^{40k}4arm-PEG-Gly-(20)-(7-ethyl-10-hydroxycamptothecin)-(10)-TBDPS (compound 8)

To a solution of ^{40k}4arm-PEGCOOH (compound 3, 1.4 g, 0.036 mmol, 1 eq.) in 14 mL of anhydrous DCM was added TBDPS-(10)-(7-ethyl-10-hydroxycamptothecin)-(20)-Gly•HCl (compound 7,207 mg, 0.29 mmol, 2.0 eq. per active site), DMAP (175 mg, 1.44 mmol, 10 eq.) and PPAC (0.85 mL of a 50% solution in EtOAc, 1.44 mmol, 10 eq.). The reaction mixture was stirred at room temperature overnight and then, evaporated under vacuum. The resulting residue was dissolved in DCM and the product was precipitated with ether and filtered. The residue was recrystallized with DMF/TPA to give the product (1.25 g). ¹³C NMR (75.4 MHz, CDCl₃): δ 7.45, 13.20, 19.39, 22.73, 26.42, 31.67, 40.21, 49.01, 66.83, 95.16, 110.02, 118.83, 125.58, 126.40, 127.53, 127.73, 129.96, 131.49, 131.76, 131.82, 135.12, 143.51, 144.78, 145.13, 146.95, 149.21, 154.61, 156.92, 16.70, 168.46, 170.30.

### Example 7

### ^{40k}4arm-PEG-Gly(20)-(7-ethyl-10-hydroxycamptothecin) (compound 9)

To compound ^{40k}4arm-PEG-Gly-(20)-(7-ethyl-10-hydroxycamptothecin)-(10)-TBDPS (compound 8, 1.25 g) was added a solution of TBAF (122 mg, 0.46 mmol, 4 eq.) in a 1:1 mixture of THF and a 0.05 M HCl solution (12.5 mL). The reaction mixture was stirred at room temperature for 4 hours and then, extracted with DCM twice. The combined organic phases were dried over MgSO₄, filtered and evaporated under vacuum. The residue was dissolved in 7 mL of DMF and precipitated with 37 mL IPA. The solid was filtered and washed with IPA. The precipitation with DMF/IPA was repeated. Finally the residue was dissolved in 2.5 mL of DCM and precipitated by addition of 25 mL of ether. The solid was filtered and dried at 40° C. in vacuum oven overnight (860 mg). ¹³ NMR (75.4 MHz, CDCl₃): δ 7.48, 13.52, 22.91, 31.67, 40.22, 49.12, 66.95, 94.82, 105.03, 118.68, 122.54, 126.37, 128.20, 131.36, 142.92, 144.20, 144.98, 147.25, 148.29, 156.44, 156.98, 166.82, 168.49, 170.39. This NMR data shows no sign of PEG-COOH which indicates that all of the COOH reacted. The loading, as determined by fluorescence detection was found to be 3.9 which is consistent with full loading of the 7-ethyl-10-hydroxycamptothecin on each of the four branches of the polymer. Repeated runs of this experiments at much larger scale yielded consistent results.

### Example 8

### Boc-(10)-(7-ethyl-10-hydroxycamptothecin) (compound 10)

To a suspension of 7-ethyl-10-hydroxycamptothecin (compound 4, 2.45 g, 1 eq.) in 250 mL of anhydrous DCM at room temperature under N₂ were added di-tert-butyl dicarbonate (1.764 g, 1.3 eq.) and anhydrous pyridine (15.2 mL, 30 eq.). The suspension was stirred overnight at room temperature. The hazy solution was filtered through celite (10 g) and the filtrate was washed with 0.5 N HCl three times (3×150 mL) and a NaHCO₃ saturated solution (1×150 ml). The solution was dried over MgSO₄ (1.25 g). The solvent was removed under vacuum at 30° C. The product was dried under vacuum at 40° C. (yield=82%, 2.525 g) ¹³C NMR (75.4 MHz, CDCl₃) d 173.53, 157.38, 151.60, 151.28, 150.02, 149.70, 147.00, 146.50, 145.15, 131.83, 127.19, 127.13, 124.98, 118.53, 113.88, 98.06, 84.26, 72.80, 66.18, 49.33, 31.62, 27.73, 23.17, 13.98, 7.90.

### Example 9

### Boc-(10)-(7-ethyl-10-hydroxycamptothecin)-(20)-Ala-Bsmoc (compound 11)

To a solution of Boc-(10)-(7-ethyl-10-hydroxycamptothecin) (compound 10, 0.85 g, 1.71 mmol) and Bsmoc-Ala (0.68 g, 2.30 mmol) in anhydrous CH₂Cl₂ (20 mL) were added EDC (0.51 g, 2.67 mmol) and DMAP (0.065 g, 0.53 mmol) at 0° C. The mixture was stirred at 0° C. for 45 min under N₂, then warmed up to room temperature. When completion of the reaction was confirmed by HPLC, the reaction mixture was washed with 1% NaHCO₃ (2×50 ml), H₂O (50 mL) and 0.1 N HCl (2×50 mL). The organic phase was dried with anhydrous MgSO₄ and filtrated. Solvent was removed under reduced pressure. The resulting solid was dried under vacuum below 40° C. overnight to give the product of 1.28 g with the yield of 95%. ¹³C NMR (75.4 MHz, CDCl₃) d: 171.16, 166.83, 157.16, 154.78, 151.59, 151.33, 149.82, 147.17, 146.68, 145.35, 145.15, 139.08, 136.88, 133.60, 131.83, 130.45, 130.40, 130.33, 127.40, 127.08, 125.32, 125.14, 121.38, 120.01, 114.17, 95.90, 84.38, 77.19, 76.64, 67.10, 56.66, 53.45, 49.96, 49.34, 31.7, 27.76, 17.94, 14.02, 7.53. ESI-MS, 786.20 [M+H]⁺.

### Example 10

### Boc-(10)-(7-ethyl-10-hydroxycamptothecin)-(20)-Ala (compound 12)

A solution of Boc-(10)-(7-ethyl-10-hydroxycamptothecin)-(20)-Ala-Bsmoc (compound 11, 4.2 g, 5.35 mmol) and 4-piperidinopiperidine (1.17 g, 6.96 mmol) in anhydrous CH₂Cl₂ (200 ml) was stirred at room temperature for 5 hours. This mixture was then washed with 0.1 N HCl (2×40 ml), followed by drying the organic layer over anhydrous MgSO₄. This solution was filtered, and the solvent was removed by vacuum distillation to yield 2.8 g of product with purity of 93%, determined by HPLC. This product was further purified by trituration with ether (3×20 ml), and then trituration with ethyl acetate (4×20 ml) to yield 1.52 g (2.70 mmol) with purity 97%. ¹³C NMR (75.4 MHz, CDCl₃) d 168.39, 166.63, 156.98, 151.20, 151.15, 149.69, 146.67, 146.56, 145.37, 144.53, 131.66, 127.13, 124.99, 119.80, 113.82, 96.15, 84.21, 77.67, 67.16, 49.48, 49.06, 31.56, 27.74, 23.14, 15.98, 13.98, 7.57.

### Example 11

### ^{40k}4arm-PEG-Ala-(20)-(7-ethyl-10-hydroxycamptothecin)-(10)-Boc (compound 13)

To anhydrous CH₂Cl₂ (100 mL) Boc-(10)-(7-ethyl-10-hydroxycamptothecin)-(20)-Ala (compound 12, 1.50 g, 2.5 mmol) and 4armPEG-COOH (compound 3, 10.01 g, 1.0 mmol) were added at room temperature. The solution was cooled to 0° C., followed by addition of EDC (0.29 g, 1.5 mmol) and DMAP (0.30 g, 2.5 mmol). The mixture was stirred at 0° C. for 1 hour under N₂. Then it was kept at room temperature overnight. The solvent was evaporated under reduced pressure. The residue was dissolved in 40 mL of DCM, and the crude product was precipitated with ether (300 mL). The wet solid resulting from filtration was dissolved in a mixture of DMF/IPA (60/240 mL) at 65° C. The solution was allowed to cool down to room temperature within 2˜3 hours, and the product was precipitated. Then, the solid was filtered and washed with ether (2×200 mL). The wet cake was dried under vacuum below 40° C. overnight to give product of 8.5 g.

### Example 12

### ^{40k}4arm-PEG-Ala-(20)-(7-ethyl-10-hydroxycamptothecin) (compound 14)

To a solution (130 mL) of 30% TFA in anhydrous CH₂Cl₂^{40k} 4arm-PEG-Ala-(20)-(7-ethyl-10-hydroxycamptothecin)-(10)-Boc (compound 13, 7.98 g) was added at room temperature. The mixture was stirred for 3 hours, or until the disappearance of starting material was confirmed by HPLC. The solvents were removed as much as possible under vacuum at 35° C. The residues were dissolved in 50 mL of DCM, and the crude product was precipitated with ether (350 mL) and filtered. The wet sold was dissolved in a mixture of DMF/IPA (50/200 mL) at 65° C. The solution was allowed to cool down to room temperature within 2˜3 hours, and the product was precipitated. Then the solid was filtered and washed with ether (2×200 mL). The wet cake was dried under vacuum below 40° C. overnight to give product of 6.7 g. ¹³C NMR (75.4 MHz, CDCl₃) d: 170.75, 169.30, 166.65, 157.00, 156.31, 148.36, 147.19, 145.03, 144.29, 143.00, 131.49, 128.26, 126.42, 122.47, 118.79, 105.10, 94.57, 78.08, 77.81, 77.20, 71.15, 70.88, 70.71, 70.33, 70.28, 70.06, 69.93, 69.57, 66.90, 49.14, 47.14, 31.53, 22.95, 17.78, 13.52, 7.46.

### Example 13

### Boc-(10)-(7-ethyl-10-hydroxycamptothecin)-(20)-Met-Bsmoc (compound 15)

To a solution of Boc-(10)-7-ethyl-10-hydroxycamptothecin (compound 10, 2.73 g, 5.53 mmol) and Bsmoc-Met (3.19 g, 8.59 mmol) in anhydrous CH₂Cl₂ (50 mL) were added EDC (1.64 g, 8.59 mmol) and DMAP (0.21 g, 1.72 mmol) at 0° C. The mixture was stirred at 0° C. for 45 minutes under N₂, then warmed up to room temperature. When completion of the reaction was confirmed by HPLC, the reaction mixture was washed with 1% NaHCO₃ (2×100 ml), H₂O (100 mL) and 0.1 N HCl (2×100 mL). The organic phase was dried with anhydrous MgSO₄ and filtrated. Solvents were removed under reduced pressure. The resulting solid was dried under vacuum below 40° C. overnight to give the product of 4.2 g with the yield of 88%. ¹³C NMR (75.4 MHz, CDCl₃) d: 170.3, 166.8, 157.1, 155.2, 151.4, 151.2, 149.7, 147.0, 146.6, 145.3, 145.1, 138.9, 136.6, 133.5, 131.7, 130.5, 130.3, 130.2, 127.3, 127.0, 125.3, 125.1, 121.2, 119.8, 114.1, 96.1, 84.3, 76.7, 67.0, 56.7, 53.5, 53.4, 49.3, 31.6, 31.0, 29.7, 27.7, 23.1, 15.4, 13.9, 7.4; ESI-MS, 846.24 [M+H]⁺.

### Example 14

### Boc-(10)-(7-ethyl-10-hydroxycamptothecin)-(20)-Met-NH₂•HCl (compound 16)

A solution of Boc-(10)-(7-ethyl-10-hydroxycamptothecin)-(20)-Met-Bsmoc (compound 15, 4.1 g, 4.85 mmol) and 4-piperidinopiperidine (1.06 g, 6.31 mmol) in anhydrous CH₂Cl₂ (200 mL) was stirred at room temperature for 5 hours. This mixture was then washed with 0.1 N HCl (2×40 ml), followed by drying the organic layer over anhydrous MgSO₄. This solution was filtered, and the solvent was removed by vacuum distillation to yield 2.8 g of product with purity of about 97%, determined by HPLC. This product was further purified by trituration with ether (3×20 ml), and then trituration with ethyl acetate (4×20 ml) to yield 1.54 g with purity of 97%. ¹³C NMR (75.4 MHz, CDCl₃) d: 167.2, 166.5, 156.9, 151.12, 150.9, 149.8, 146.3, 145.9, 145.8, 144.9, 131.3, 127.2, 127.0, 125.1, 119.6, 113.8, 96.7, 84.3, 78.2, 67.0, 60.4, 52.2, 49.4, 31.4, 29.6, 29.1, 27.7, 23.2, 15.1, 13.9, 7.7.

### Example 15

### ^{40k}4arm-PEG-Met-(20)-(7-ethyl-10-hydroxycamptothecin)-(10)-Boc (compound 17)

To an anhydrous CH₂Cl₂ (80 mL) solution, Boc-(10)-(7-ethyl-10-hydroxycamptothecin)-(20)-Met (compound 16, 1.48 g, 2.25 mmol) and 4arm-PEG-COOH (compound 3, 9.0 g, 0.9 mmol) were added at room temperature. The solution was cooled to 0° C. followed by addition of EDC (0.26 g, 1.35 mmol) and DMAP (0.27 g, 2.25 mmol). The mixture was stirred at 0° C. for 1 hour under N₂. Then it was kept at room temperature overnight. The reaction mixture was diluted with 70 ml of CH₂Cl₂, extracted with 30 ml of 0.1 N HCl/1 M NaCl aqueous solution. After the organic layer was dried with MgSO₄, the solvent was evaporated under reduced pressure. The residue was dissolved in 40 mL of CH₂Cl₂, and the crude product was precipitated with ether (300 mL). The wet solid resulting from filtration was dissolved in 270 mL of DMF/IPA at 65° C. The solution was allowed to cool down to room temperature within 2˜3 hours, and the product was precipitated. Then the solid was filtered and washed with ether (2×400 mL). The above crystallization procedure in DMF/IPA was repeated. The wet cake was dried under vacuum below 40° C. overnight to give product of 7.0 g. ¹³C NMR (75.4 MHz, CDCl₃) d: 169.8, 169.6, 166.5, 156.9, 151.2, 151.1, 149.9, 147.0, 146.6, 145.0, 131.7, 127.1, 126.8, 124.9, 119.7, 113.8, 95.5, 84.1, 70.1, 69.9, 66.9, 50.7, 49.2, 31.5, 31.2, 29.6, 27.6, 231, 15.3, 13.9, 7.5.

### Example 16

### ^{40k}4arm-PEG-Met-(20)-(7-ethyl-10-hydroxycamptothecin) (compound 18)

To a solution of 30% TFA in anhydrous CH₂Cl₂ (100 mL), dimethyl sulfide (2.5 mL) and 4arm-PEG-Met-(20)-(7-ethyl-10-hydroxycamptothecin)-(10)-Boc (compound 17, 6.0 g) were added at room temperature. The mixture was stirred for 3 hours, or until disappearance of starting material was confirmed by HPLC. Solvents were removed as much as possible under vacuum at 35° C. The residues were dissolved in 50 mL of CH₂Cl₂, and the crude product was precipitated with ether (350 ml), and filtered. The wet solid was dissolved in a mixture of DMF/IPA (60/300 mL) at 65° C. The solution was allowed to cool down to room temperature within 2˜3 hours, and the product was precipitated. Then the solid was filtered and washed with ether (2×200 mL). The wet cake was dried under vacuum below 40° C. overnight to give product of 5.1 g. ¹³C NMR (75.4 MHz, CDCl₃) d : 169.7, 166.6, 157.0, 156.3, 148.4, 147.3, 145.0, 144.4, 142.9, 131.5, 128.3, 126.4, 122.5, 118.7, 105.2, 94.7, 78.1, 67.0, 50.7, 49.2, 31.6, 31.3, 29.7, 23.0, 15.3, 13.5, 7.5; Ratio of 7-ethyl-10-hydroxycamptothecin to PEG: 2.1% (wt).

### Example 17

### Boc-(10)-(7-ethyl-10-hydroxycamptothecin)-(20)-Sar-Boc (compound 19)

Boc-Sar-OH (432 mg, 2.287 mmol) was added to a solution of Boc-(10)-(7-ethyl-10-hydroxycamptothecin) (compound 10, 750 mg, 1.52 mmol) in 75 mL of DCM and cooled to 0° C. DMAP (432 mg, 2.287 mmol) and EDC (837 mg, 0.686 mmol) were added and the reaction mixture was stirred from 0° C.-room temperature for 1.5 hours. Reaction mixture was then washed with 0.5% NaHCO₃ (75 mL×2), with water (75 ml×2) and finally washed with 0.1 N HCl (75 mL×1). The methylene chloride layer was dried over MgSO₄ and the solvent was evaporated under vacuum and dried. Yield=0.900 mg. (89%). The structure was confirmed by NMR.

### Example 18

### 7-ethyl-10-hydroxycamptothecin-(20)-Sar•TFA (compound 20)

Boc-(10)-(7-ethyl-10-hydroxycamptothecin)-(20)-Sar-Boc (compound 19, 900 mg, 1.357 mmol) was added to a solution of 4 mL TFA and 16 mL DCM, and stirred at room temperature for 1 hour. The reaction mixture was evaporated with toluene at 30° C. The residue was dissolved in 10 mL CHCl₃ and precipitated with ethyl ether. The product was filtered and dried. Yield 700 mg (1.055 mmol, 78%). ¹³C NMR (67.8 MHz, CDCl₃) δ 168.26, 167.07, 158.84, 158.71, 148.82, 147.94, 147.22, 146.34, 144.04, 131.18, 130.08, 128.97, 124.46, 119.78, 106.02, 97.23, 79.84, 79.34, 66.87, 50.84, 49.86, 31.81, 23.94, 15.47, 13.84, 8.08.

### Example 19

### TBDMS-(10)-(7-ethyl-10-hydroxycamptothecin)-(20)-Sar•HCl (compound 21)

A solution of the 7-ethyl-10-hydroxycamptothecin-(20)-Sar•TFA (compound 20, 2.17 g, 3.75 mmol, 1 eq.) in anhydrous DMF (30 mL) was diluted with 200 mL of anhydrous DCM. Et₃N (2.4 mL, 17.40 mmol, 4.5 eq.) was added followed by TBDMSCl (2.04 g, 13.53 mmol, 3.5 eq.). The reaction mixture was stirred at room temperature until HPLC showed disappearance of the starting material (approximately 1 hour). The organic layer was washed with 0.5% NaHCO₃ twice, water once, and a 0.1 N HCl solution saturated with brine twice; and then dried over MgSO₄. After filtration and evaporation of the solvent under vacuum, the resulting oil was dissolved in DCM. Addition of ether gave a solid that was filtered using a fine or medium buchner funnel (2.00 g, 87% yield). HPLC of the solid showed 96% purity. ¹H NMR and ¹³C NMR confirmed the structure. ¹H NMR (300 MHz, CD₃OD): δ 0.23 (6H, s), 0.96 (9H, s), 0.98 (3H, t, J=7.3 Hz), 1.30 (3H, t, J=7.6 Hz), 2.13-2.18 (2H, m), 2.67 (3H, s), 3.11 (2H, q, J=7.6 Hz), 4.10 (1H, d, J=17.6 Hz), 4.22 (1H, d, J=17.6 Hz), 5.23 (2 H, s), 5.40 (1H, d, J=16.7 Hz), 5.55 (1H, d, J=16.7 Hz), 7.32 (1H, s), 7.38-7.43 (2H, m), 8.00 (1H, d, J=9.1 Hz). ¹³C NMR (75.4 MHz, CD₃OD): δ-4.14, 8.01, 14.10, 19.30, 23.98, 26.16, 31.78, 33.52, 49.46, 50.95, 67.66, 79.80, 97.41, 111.96, 119.99, 127.75, 129.28, 129.67, 131.57, 145.24, 146.86, 147.16, 148.02, 150.34, 156.69, 158.72, 167.02, 168.27.

### Example 20

### ^{40K} 4arm-PEG-Sar-(20)-(7-ethyl-10-hydroxycamptothecin)-(10)-TBDMS (compound 22)

To a solution of ^{40K}4arm-PEG-COOH (compound 3, 10 g, 0.25 mmol, 1 eq.) in 150 mL of anhydrous DCM was added a solution of TBDMS-(10)-(7-ethyl-10-hydroxycamptothecin)-Sar•HCl (compound 21, 1.53 g, 2.5 mmol, 2.5 eq.) in 20 mL of anhydrous DMF and the mixture was cooled to 0° C. To this solution were added EDC (767 mg, 4 mmol, 4 eq.) and DMAP (367 mg, 3 mmol, 3 eq.) and the reaction mixture was allowed to warm to room temperature slowly and stirred at room temperature overnight. Then, the reaction mixture was evaporated under vacuum and the residue was dissolved in a minimum amount of DCM. After addition of ether, solid was formed and filtered under vacuum. The residue was dissolved in 30 mL of anhydrous CH₃CN and precipitated by addition of 600 mL IPA. The solid was filtered and washed with IPA and ether to give the product (9.5 g). The structure was confirmed by NMR.

### Example 21

### ^{40K}4arm-PEG-Sar-(20)-(7-ethyl-10-hydroxycamptothecin) (compound 23)

Method A. ^{40K}4arm-PEG-Sar-(20)-(7-ethyl-10-hydroxycamptothecin)-(10) TBDMS (compound 22) was dissolved in a 50% mixture of TFA in H₂O (200 mL). The reaction mixture was stirred at room temperature for 10 hours and then, diluted with 100 mL of H₂O and extracted with DCM (2×300 mL). The combined organic phases were washed with H₂O (2×100 mL), dried over MgSO₄, filtered and evaporated under vacuum. The residue was dissolved in 100 mL of anhydrous DMF gently heated with a heat gun and precipitated by slow addition of 400 mL DMF. The solid was filtered and washed with 20% DMF in IPA and ether. The solid was dissolved in DCM and precipitated with ether (6.8 g). The structure was conformed by NMR.
Method B. ^{40K} 4arm-PEG-Sar-(20)-(7-ethyl-10-hydroxycamptothecin)-(10)-TBDMS (1 g) was dissolved in 10 mL of a 1N HCl solution. The reaction mixture was stirred at room temperature for 1 hour (checked by HPLC) and then extracted with DCM (2×40 mL). The organic layers were dried over MgSO₄, filtered and evaporated under vacuum. The resulting bright yellow residue was dissolved in 10 mL of DMF (slightly heated with a heat gun) and then 40 mL of IPA were added. The resulting solid was filtered and dried overnight at 40° C. in a vacuum oven. The structure was confirmed by NMR.

### Biological Data

### Example 22

### Toxicity Data

A maximum tolerated dose (“MTD”) of four-arm PEG conjugated 7-ethyl-10-hydroxycamptothecin (compound 9) as prepared by Example 7, supra, was studied using nude mice. Mice were monitored for 14 days for mortality and signs of illness and sacrificed when body weight loss was >20% of the pretreatment body weight.

Table 2, below, shows the maximum tolerated dose (“MTD”) of each compound for both single dose and multiple dose administration. Each dose for multiple dose administration was given mice every other day for 10 days and the mice were observed for another 4 days, thus for total 14 days.

TABLE 2
MTD Data in Nude Mice

| | Dose Level | | |
|---|---|---|---|
| Compound | (mg/kg) | Survival/Total | Comments |
| Compound 9 | 25 | 5/5 | |
| Single dose | 30 | 5/5 | |
| | 35 | 4/5 | Mouse euthanized due to |
| | | | >20% body weight loss |
| Compound 9 | 10 | 5/5 | |
| Multiple dose* | 15 | 3/5 | Mice euthanized due to |
| | | | >20% body weight loss |
| | 20 | 0/5 | Mice euthanized due to |
| | | | >20% body weight loss |

The MTD found for 4arm-PEG-Gly-(7-ethyl-10-hydroxycamptothecin) (compound 9) was 30 mg/kg when given as single dose, and 10 mg/kg when given as multiple dose (q2d×5).

### Example 23

### Properties of PEG Conjugates

Table 3, below, shows solubility of four different PEG-(7-ethyl-10-hydroxycamptothecin) conjugates in aqueous saline solution. All four PEG-(7-ethyl-10-hydroxycamptothecin) conjugates showed good solubility of up to 4 mg/mL equivalent of 7-ethyl-10-hydroxycamptothecin. In human plasma, 7-ethyl-10-hydroxycamptothecin was steadily released from the PEG conjugates with a doubling time of 22 to 52 minutes and the release appeared to be pH and concentration dependent as described in the following EXAMPLE 24.

TABLE 3
Properties of PEG-7-ethyl-10-hydroxycamptothecin Conjugates

| | Solubility in | t_{1/2} (min) in Human | Doubling Time in Plasma (min)^{c} | | |
|---|---|---|---|---|---|
| Compound | Saline (mg/mL)^{a} | Plasma^{b} | Human | Mouse | Rat |
| Compound 9 | 180 | 12.3 | 31.4 | 49.5 | 570 |
| (Gly) | | | | | |
| Compound 12 | 121 | 12.5 | 51.9 | 45.8 | 753 |
| (Ala) | | | | | |
| Compound 23 | ND | 19.0 | 28.8 | 43.4 | 481 |
| (Sar) | | | | | |
| Compound 18 | 142 | 26.8 | 22.2 | 41.9 | 1920 |
| (Met) | | | | | |

^{a}7-ethyl-10-hydroxycamptothecin is not soluble in saline.
^{b}PEG conjugate half life.
^{c}7-ethyl-10-hydroxycamptothecin formation rate from conjugates.

20 PEG-7-ethyl-10-hydroxycamptothecin conjugates show good stability in saline and other aqueous medium for up to 24 hours at room temperature.

### Example 24

### Effects of Concentration and pH on Stability

Based on our previous work, acylation at the 20-OH position protects the lactone ring in the active closed form. The aqueous stability and hydrolysis properties in rat and human plasma were monitored using UV based HPLC methods. 4armPEG-Gly-(7-ethyl-10-hydroxycamptothecin) conjugates were incubated with each sample for 5 minutes at room temperature.

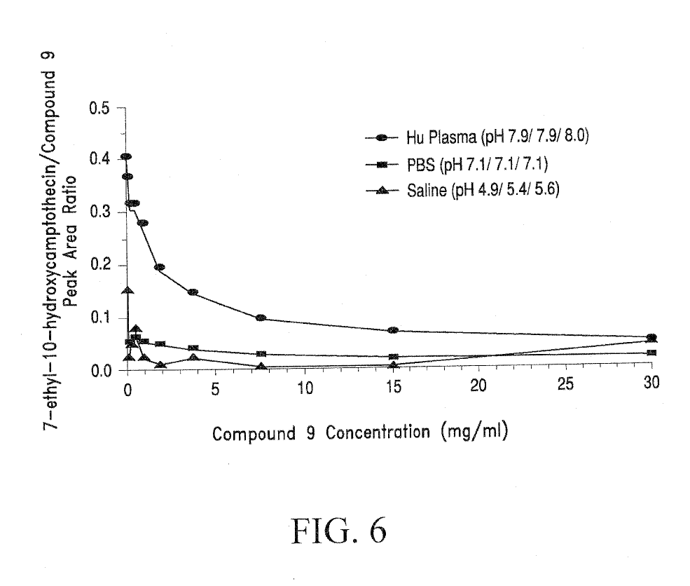

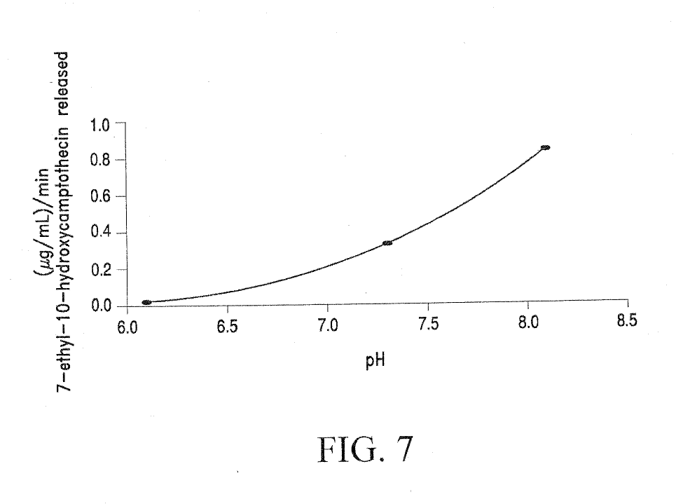

Stability of PEG-7-ethyl-10-hydroxycamptothecin conjugates in buffer was pH dependent. FIG. 6 shows 4armPEG-Gly-(7-ethyl-10-hydroxycamptothecin) stability in various samples. FIG. 7 shows that rate of 7-ethyl-10-hydroxycamptothecin release from PEG-Gly-(7-ethyl-10-hydroxycamptothecin) increases with increased pH.

### Example 25

### Pharmacokinetics

Tumor free Balb/C mice were injected with a single injection of 20 mg/kg 4armPEG-Gly-(7-ethyl-10-hydroxycamptothecin) conjugates. At various time points mice were sacrificed and plasma was analyzed for intact conjugates and released 7-ethyl-10-hydroxycamptothecin by HPLC. Pharmacoknetic analysis was done using non-compartmental analysis (WinNonlin). Details are set forth in Table 4, below.

TABLE 4
Pharmacokinetic Data

| | | 7-ethyl-10-hydroxy- |
|---|---|---|
| | | camptothecin Released |
| Parameter | Compound 9 | from Compound 9 |
| AUC (h * μg/mL) | 124,000 | 98.3 |
| Terminal t_{1/2} (Hr) | 19.3 | 14.2 |
| Cₘₐₓ (μg/mL) | 20,500 | 13.2 |
| CL (mL/hr/kg) | 5.3 | 202 |
| Vss (mL/kg) | 131 | 3094 |

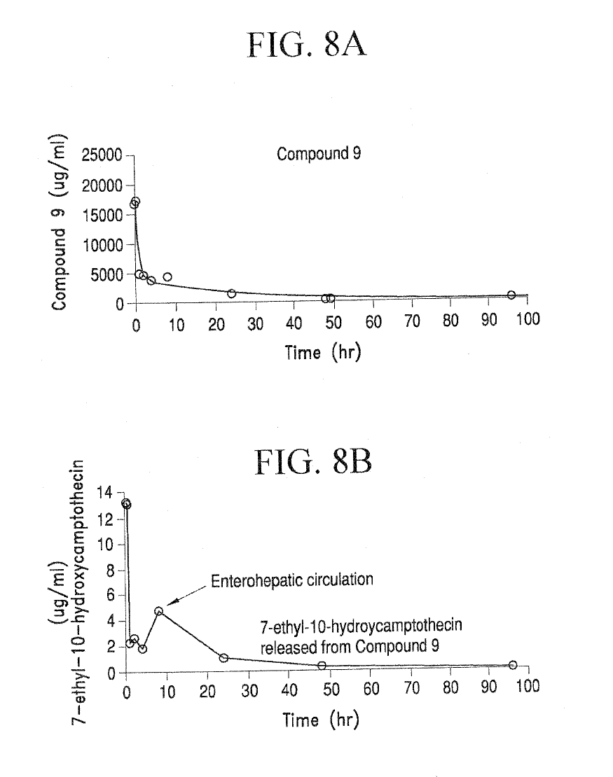

As shown in FIGS. 8A and 8B, PEGylation of 7-ethyl-10-hydroxycamptothecin allows long circulation half life and high exposure to native drug 7-ethyl-10-hydroxycamptothecin. Enterohepatic circulation of 4armPEG-Gly-(7-ethyl-10-hydroxycamptothecin) conjugates was observed. The pharmacokinetic profile of PEG-Gly-(7-ethyl-10-hydroxycamptothecin) in mice was biphasic showing a rapid plasma distribution phase during the initial 2 hours followed by a 18-22 hours terminal elimination half-life for the conjugate and a concomitant 18-26 hours terminal elimination half-life for 7-ethyl-10-hydroxycamptothecin.

Additionally, pharmacokinetic profiles of 4arm PEG-Gly-(7-ethyl-10-hydroxycamptothecin) were investigated in rats. In rats, does levels of 3, 10 and 30 mg/kg (7-ethyl-10-hydroxycamptothecin equivalent) were used. The pharmacokinetic profiles in rats were consistent with those of mice.

In rats, 4arm PEG-Gly-(7-ethyl-10-hydroxycamptothecin) showed a biphasic clearance from the circulation with an elimination half life of 12-18 hours in rats. 7-ethyl-10-hydroxycamptothecin released from 4armPEG-Gly-7-ethyl-10-hydroxycamptothecin conjugates had an apparent elimination half life of 21-22 hours. The maximum plasma concentration (Cₘₐₓ) and area under the curve (AUC) increased in a dose dependent manner in rats. The apparent half life of released 7-ethyl-10-hydroxycamptothecin from 4armPEG-Gly conjugates in mice or rats is significantly longer than the reported apparent half life of released 7-ethyl-10-hydroxycamptothecin from CPT-11 and the exposure of released 7-ethyl-10-hydroxycamptothecin from 4arm PEG-Gly-(7-ethyl-10-hydroxycamptothecin) is significantly higher than the reported exposure of released 7-ethyl-10-hydroxycamptothecin from CPT-11. The clearance of the parent compound was 0.35 mL/hr/kg in rats. The estimated volume of distribution at steady state (Vss) of the parent compound was 5.49 mL/kg. The clearance of the released 7-ethyl-10-hydroxycamptothecin was 131 mL/hr/kg in rats. The estimated Vss of released 7-ethyl-10-hydroxycamptothecin was 2384 mL/kg in rats. Enterohepatic circulation of released 7-ethyl-10-hydroxycamptothecin was observed both in mice and rats.

### Example 26

### Therapeutic Efficacy in RAS-Associated Human Lung Carcinoma Xenografted Mice

The objective of this study was to evaluate the efficacy of 4armPEG-Gly-(7-ethyl-10-hydroxycamptothecin) conjugates (compound 9) prepared according to Example 7, in a Kras mutant xenograft model of human lung cancer (Calu 6).

### Test Agents:

Compound 9, provided as a powder, storage at −20° C.

Erbitux® (Imclone), Lot Number: 07C004638, provided as a liquid, 2 mg/ml, storage at 4° C.

Camptosar® (Pfizer) CPT-11, Lot Number: OAMDS, Expiration September 2009, provided as a liquid, 20 mg/ml, storage at 4° C. after opening.

### Cells, Animals, Model

Calu-6 Human Lung Carcinoma cells were maintained EMEM (with 1% Penicillin/Streptomycin and 10% FBS. The cell lines were cultured at 37° C. in a 5% CO₂ incubator.

As shown by the Sanger Institute Catalog of Somatic Mutations (see website at http://www.sanger.ac.uk/), incorporated by reference herein, Calu-6 cells have the following Kras mutations.
Kras protein: Q61 K.
KRAS cDNA: 586C→T.

Female athymic nude-Foxn1^{NU/}Foxn1⁺ mice (Harlan Indianapolis, Ind.) were housed at the University of Medicine and Dentistry of New Jersey (UMDNJ) vivarium facility (Piscataway, N.J.). Mice received food and water ad libitum. Following a 10-day acclimation period (at 5-6 weeks of age) mice were injected with 5×10⁶ Calu-6 cells/mouse subcutaneously. The study was performed in two parts. One part of the study aimed at comparing the efficacy of compound 9, CPT-11 or C225 as single agent therapies. For this part, when tumors reached an average volume of 170 mm³, mice were divided into following groups; compound 9 at its single dose MTD (30 mg/kg qd×1), CPT-11 at its single dose MTD (80 mg/kg qd×1), compound 9 at its multiple dose MTD (10 mg/kg q2d×5), CPT-11 at its multiple dose MTD (40 mg/kg q2d×5) or Erbitux® 1 mg/mouse (2×/week for 5 doses—this is two doses per week over a 2.5 week time period).

Body weight and tumor volume were monitored twice weekly. The tumor volume for each mouse was determined by measuring two dimensions with calipers and calculated using the formula: tumor volume=(length×width²)/2). The animals were monitored daily for any signs of illness and will be weighed weekly. Mice were assessed for illness and terminated at signs of illness that include, but not limited to, paralysis of hind limbs, scruffy coat, lethargy, weight loss of =20%. The observation period lasted to a maximum of 5 times the median survival time of control animals; after which the mice were euthanized by CO₂ inhalation.

### Results

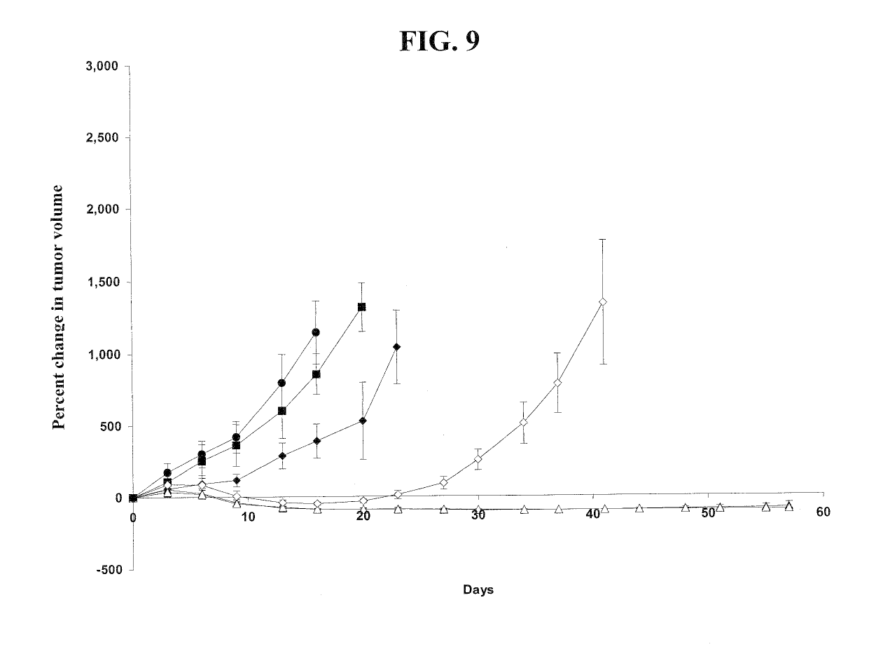

As seen from FIG. 9, mice treated with compound 9 either as a single dose or in multiple doses had significantly improved therapeutic efficacy compared to other groups.

FIG. 9 Legend:
(▪) indicates saline;
(▴) indicates compound 9 at 30 mg/kg/qd×1 (the curve is behind (Δ) curve);
(♦) indicates CPT-11 at 80 mg/kg qd×1;
(Δ) indicates compound 9 at 10 mg/kg q2d×5;
(⋄) indicates CPT-11 at 40 mg/kg q2d×5;
() indicates C-225.

Compound 9, either as single agent or in multiple doses, had 99% tumor growth inhibition (“TGI”). Multiple dose treatment of compound 9 led to cures (no signs of visible tumor) of all the animals in the trial. In contrast, C225 or Erbitux® was completely ineffective as single agent therapy in this model. Therefore, as seen in the clinic, C225 fails in KRAS mutant cancers. CPT-11 as a single agent had a modest 52% TGI and all animals had to be sacrificed by about day 22-24 due to excessive tumor burden. On the other hand, CPT-11 as multiple doses resulted in 95% TGI; however again animals relapsed by day 40-45, and had to be sacrificed due to their tumor burden. Only animals treated with compound 9 were cured in the study, thus suggesting that treatment with compound 9 may be an effective way to treat KRAS mutant cancers.

TABLE 5
Summary of results.

| | | Mean | | | |
|---|---|---|---|---|---|
| | | Tumor | | | Observed |
| | | Volume | % | % | cures |
| Grps | Test Compound | (mm³) | T/C | TGI | (#/n) |
| 1 | Saline | 2414 | — | — | 0/8 |
| 2 | compound 9 30 mg/kg qdx1 | 19 | 1 | 99 | 6/8 |
| 3 | compound 9 10 mg/kg q2dx5 | 17 | 1 | 99 | 8/8 |
| 4 | CPT-11 80 mg/kg qdx1 | 1149 | 48 | 52 | 0/8 |
| 5 | CPT-11 40 mg/kg q2dx5 | 110 | 5 | 95 | 0/8 |
| 6 | C225 1 mg/mouse | — | — | — | 0/8 |

Cures taken from day 57 data otherwise values calculated from day 20 data

### Example 27

### Therapeutic Efficacy in Ras-Associated Human Lung Carcinoma Xenografted Mice Refractory to EGFR Antagonist

The second part of the study aimed at evaluating the efficacy of compound 9 or CPT-11 in Erbitux® failed tumors. For this part, when tumors reached an average volume of 170 mm³, mice received an initial two dose challenge of Erbitux® 1 mg/mouse (2×/week). Mice were followed and at this stage, mice were divided into 3 groups that received either compound 9, 10 mg/kg (q2d×5) or CPT-11 40 mg/kg (q2d×5) or Erbitux® 1 mg/mouse (2×/week for 3 additional doses—for a treatment period of 1.5 weeks). A negative control group received saline placebo.

The second part of the study confirmed that compound 9 is effective in KRAS mutant xenografts that fail C225 therapy. As described in the methods section, above, animals were first treated with C225, and when they failed C225 therapy, they were divided into 3 groups to be treated with compound 9, CPT-11 or C225.

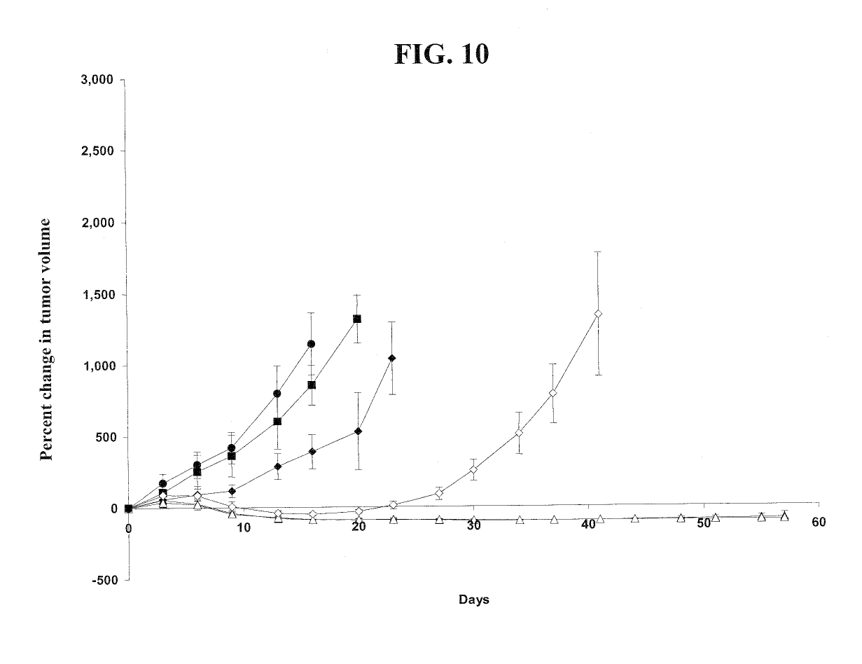

As summarized by FIG. 10, both CPT-11 and compound 9 were initially effective in the C225 failed population, however CPT-11 treatment eventually failed, and test animals, showing tumor growth. The compound 9 treated animals, on the other hand, exhibited curing of 5 of the 8 animals in the trial.

FIG. 10 Legend:
(▪) indicates saline;
(Δ) indicates compound 9 at 10 mg/kg q2d×5 with C-225 challenge;
(⋄) indicates CPT-11 at 40 mg/kg q2d×5 with C-225 challenge;
() indicates C-225

Therefore, this study confirms, that compound 9 therapy is an effective treatment for KRAS mutant cancers that failed C225 treatment.

TABLE 6
Summary of results.

| | | Mean | | | |
|---|---|---|---|---|---|
| | | Tumor | | | Observed |
| | | Volume | % | % | cures |
| Grps | Test Compound | (mm³) | T/C | TGI | (#/n) |
| 7 | C225 1 mg/mouse | — | — | — | 0/8 |
| | (after C-225 challenge) | | | | |
| 8 | compound 9 10 mg/kg q2dx5 | 242 | 10 | 90 | 5/8 |
| | after C-225 challenge | | | | |
| 9 | CPT-11 40 mg/kg q2dx5 | 376 | 16 | 84 | 0/8 |
| | after C-225 challenge | | | | |

Cures taken from day 57 data otherwise values calculated from day 20 data

### Example 28

### Therapeutic Efficacy in Ras-Associated Human Pancreatic Cancer Xenografted Mice

The efficacy of compound 9 was evaluated in a Kras mutant pancreatic cancer xenograft model (MiaPaCa-2). As shown by the Sanger Institute Catalog of Somatic Mutations (see website at http://www.sanger.ac.uk), incorporated by reference herein, the MiaPaCA-2 cells exhibit the following KRAS mutations.
Kras protein: G12C.
KRAS cDNA: 34G→T.

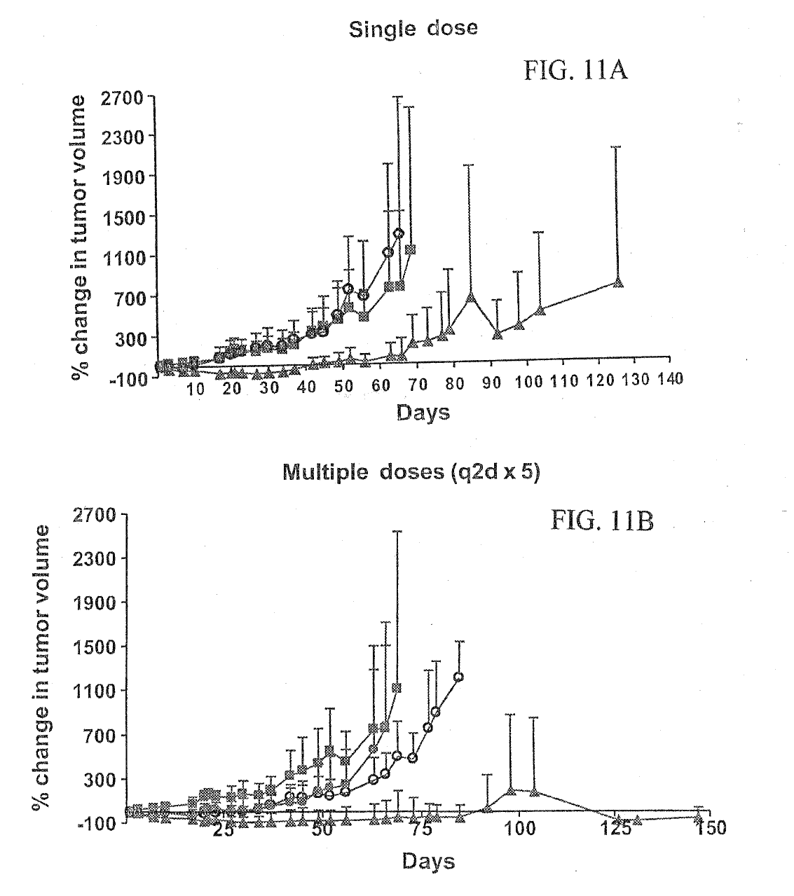

Female athymic nude mice (6-10 mice per group), were inoculated s.c. with 2.5×10⁶ MiaPaCA-2 pancreatic cells. When tumors reached ˜100 mm³ mice were treated with either a single (FIG. 11A) or multiple (q2d×5) injections (FIG. 11B). For the single dose treatment regimen, mice were treated with saline (▪), compound 9 (▴) at a single dose (30 mg/kg qd×1), or CPT-11 (◯) at a single dose (80 mg/kg qd×1). For the multiple dose treatment regimen, mice were given saline (▪), compound 9 (▴) at 10 mg/kg q2d×5, CPT-11 (◯) at 40 mg/kg q2d×5 or 10 mg/kg q2d×5 which is equivalent MTD of compound 9.

On this cell line, in vitro compound 9 was 614-fold more potent than CPT-11. Treatment with a single MTD of compound 9 resulted in 71% TGI (on Day 69) and 100% survival of animals. However, a single dose of CPT-11 given at the MTD had no effect on tumor growth. Multiple-dose treatment of compound 9 caused 95% TGI, and by Day 147 (last day of study), 66% of animals were cured (no evidence of tumors by gross observation). In contrast, multiple-dose CPT-11 treatment resulted in 47% and 20% TGI when dosed at MTD or at corresponding dose level as compound 9 (10 mg/kg, q2d×5), respectively.

### Example 29

### Therapeutic Efficacy in Ras-Associated Human Colorectal Cancer Xenografted Mice

The efficacy of compound 9 in treating a Kras mutant xenograft model of human colorectal cancer (SW480) was evaluated.

### Materials

Compound 9, provided as a powder, storage at −20° C.
Erbitux® (Imclone), Lot Number: 07C004638, provided as a liquid, 2 mg/ml, storage at 4° C.
Camptosar® (Pfizer) CPT-11, Lot Number: OAMDS, Expiration September 2009, provided as a liquid, 20 mg/ml, storage at 4° C. after opening.

### Cells, Animals, Model

SW480 Human colorectal carcinoma cells were maintained in Leibovitz's L-15 media (with 1% Penicillin/Streptomycin and 10% FBS). SW480 cells have been shown to have a mutated Ras gene by Capon et al., Nature 304(11):407-513. The cell lines were cultured at 37° C. in a 5% CO₂ incubator. Female athymic nude-Foxn1^{NU/}Foxn1⁺ mice (Harlan Indianapolis, Ind.) were housed at the UMDNJ vivarium facility (Piscataway, N.J.). Mice received food and water ad libitum. Following a 31-day acclimation period (at 11-12 weeks of age) mice were injected with 5×10⁶ SW480 cells/mouse subcutaneously.

Part 1 of the study compared the efficacy of compound 9, CPT-11 or C225 as single agent therapies. For this part, when tumors reached an average volume of 95 mm³, mice were divided into following groups; compound 9 at its single dose MTD (30 mg/kg qd×1), CPT-11 at its single dose MTD (80 mg/kg qd×1), compound 9 at its multiple dose MTD (10 mg/kg q2d×5), CPT-1 at its multiple dose MTD (40 mg/kg q2d×5), Erbitux® 1 mg/mouse (2×/week for 5 doses) or compound 91 mg/kg (q2d×5).

Body weight and tumor volume were monitored twice weekly. The tumor volume for each mouse was determined by measuring two dimensions with calipers and calculated using the formula: tumor volume=(length×width²)/2. The animals were monitored daily for any signs of illness and weighed weekly. Mice were assessed for illness and terminated at signs of illness that included, but not limited to, paralysis of hind limbs, scruffy coat, lethargy, weight loss of =20%.

### Results

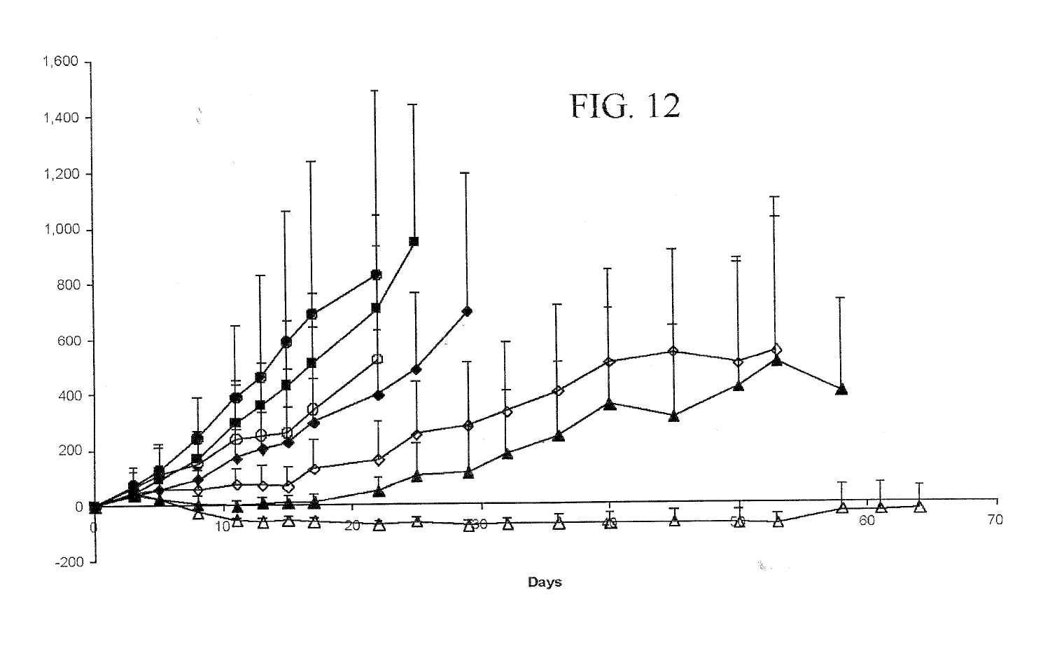

As seen from FIG. 12, mice treated with compound 9 either as a single dose or in multiple doses had significantly improved therapeutic efficacy compared to other groups. Compound 9 (on day 22) was administered either as single agent or in multiple doses, which resulted in a TGI of over 81% or 96% respectively. Concurrently, CPT-11 was administered as a single agent. These animals exhibited a modest 47% TGI, while the multiple dose regime resulted in 65% TGI. All other groups have relapsed or have been euthanized due to tumor burden.

TABLE 7
Summary of results. (Data taken from Day 22).

| | | Mean | | | |
|---|---|---|---|---|---|
| | | Tumor | | | |
| | | Volume | Percent | | |
| Grps | Compound | (TV) (mm³) | Change in TV | % T/C* | % TGI |
| 1 | Saline | 861.04 | 709.52 | — | — |
| | | (571.54) SD | (332.71) SD | | |
| 2 | Compound 9 30 mg/kg qdx1 | 159.37 | 52.15 | 22.40 | 81.49 |
| | | (79.20) SD | (50.72) SD | | |
| 3 | Compound 9 10 mg/kg q2dx5 | 29.68 | −67.29 | 4.52 | 96.55 |
| | | (13.78) SD | (14.02) SD | | |
| 4 | CPT-11 80 mg/kg qdx1 | 460.42 | 397.75 | 66.60 | 46.53 |
| | | (257.15) SD | (235.07) SD | | |
| 5 | CPT-11 40 mg/kg q2dx5 | 303.47 | 163.62 | 44.52 | 64.76 |
| | | (217.10) SD | (135.61) SD | | |
| 6 | C-225 1 mg/mouse | 870.16 | 832.75 | 129.88 | −1.06 |
| | | (613.08) SD | (662.87) SD | | |
| 7 | Compound 9 1 mg/kg | 544.54 | 524.57 | 67.79 | 36.76 |
| | | (361.71) SD | (409.98) SD | | |

*T/C is tumor volume of treated group divided by tumor volume of control group.

### Example 30

### Therapeutic Efficacy in Ras-Associated Human Colorectal Cancer Xenografted Mice Refractory to EGFR Antagonist

Part 2 of the study compared the efficacy of compound 9 or CPT-11 in Erbitux® failed tumors. For this part, human colorectal cancer (SW480) was established in mice as described in Example 29. When tumors reached an average volume of 95 mm³, mice received an initial three dose challenge of Erbitux® 1 mg/mouse (2×/week—for a one week treatment period). Mice were followed and at this stage, mice were divided into 3 groups that received either compound 910 mg/kg (q2d×7) or CPT-11 40 mg/kg (q2d×7) or Erbitux® 1 mg/mouse (2×/week for 6 additional doses). A negative control group received saline placebo.

Body weight and tumor volume were monitored twice weekly. The animals were monitored daily for any signs of illness and weighed weekly. Mice were assessed for illness and terminated at signs of illness that included, but not limited to, paralysis of hind limbs, scruffy coat, lethargy, weight loss of =20%.

### Results

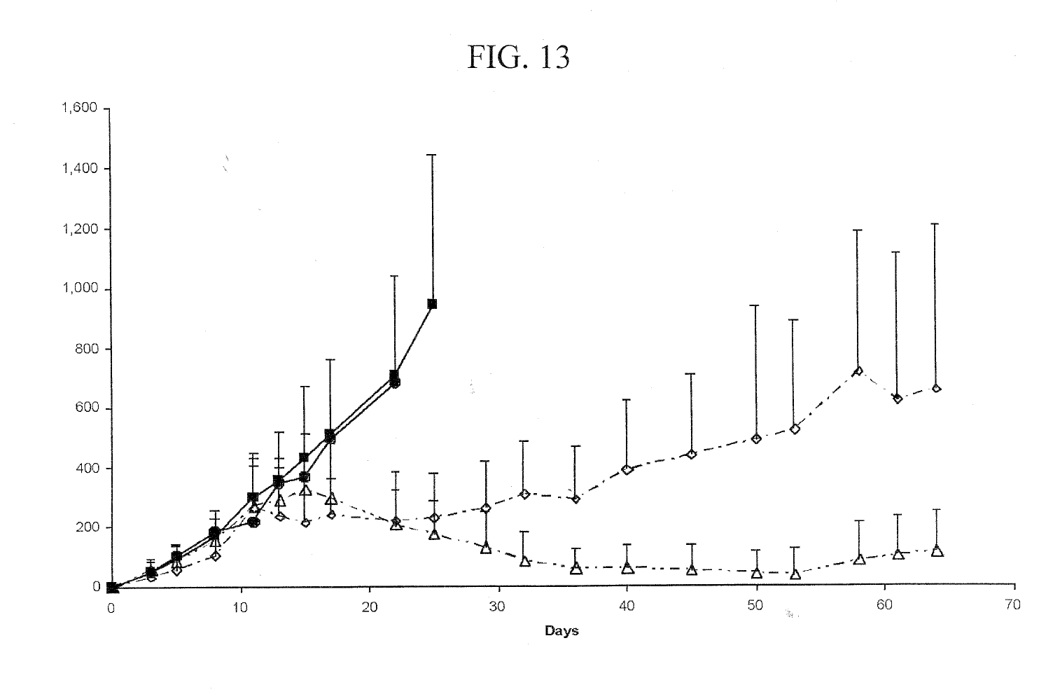

As of day 64 of the study, only compound 9 10 mg/kg q2d×7 maintained a mean tumor volume below baseline values (FIG. 13). All other groups have relapsed or have been euthanized due to tumor burden.

TABLE 8
Summary of results. (Data taken from Day 22).

| | | Mean | | | |
|---|---|---|---|---|---|
| | | Tumor | | | |
| | | Volume | Percent | | |
| Grps | Compound | (TV) (mm³) | Change in TV | % T/C* | % TGI |
| 8 | Compound 9 10 mg/kg q2dx7 | 283.54 | 208.54 | 34.24 | 67.07 |
| | (after C-225 challenge) | (153.08) SD | (172.94) SD | | |
| 9 | CPT-11 40 mg/kg q2dx7 after | 298.07 | 219.31 | 43.65 | 65.38 |
| | C-225 challenge | (105.26) SD | (106.86) SD | | |
| 10 | C-225 1 mg/ms | 768.10 | 684.74 | 85.87 | 10.79 |
| | after C-225 challenge | (453.63) SD | (549.08) SD | | |

*T/C is tumor volume of treated group divided by tumor volume of control group.

### Example 31

### Therapeutic Efficacy of Ras-Associated Human Colorectal Cancer Xenografted Mice with High EGFR Expression

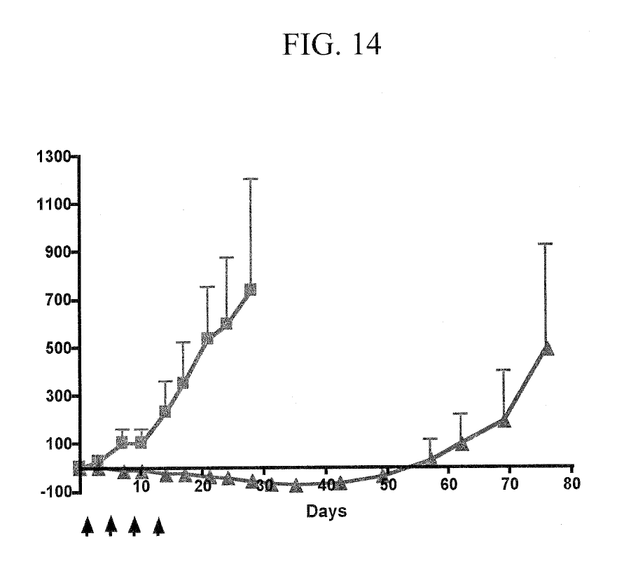

Therapeutic efficacy of compound 9 was evaluated in additional Ras associated human colorectal cancer xenografted mice. HCT1116 and SW480 cell lines are known to express EGFR in moderate to high levels, as compared to low expresser Calu6 human lung cancer line. HCT-116 Kras mutant human colorectal xenografts were established in female nude mice via injection of 5×10⁶ cells/mouse sc. When tumors reached a mean volume of 367.7±123.0 mm³, mice received either saline or compound 9 10 mg/kg 2×/week×2 weeks i.v. By day 28 (the last day of control survival) compound 9 resulted in a TGI of 93%. Therefore, as illustrated by FIG. 14, compound 9 is effective in regressing Kras mutant xenografts.

As confirmed by Examples 29, 30 and 31, the present invention provides means for treating cancer with Ras mutation and high expression of EGFR.

Numerous references are cited hereinabove, all of which are incorporated by reference herein in their entireties.

We claim:

1. A method of treating a Ras associated cancer in a mammal comprising:
(i) determining the presence of a mutation in a RAS gene in a cancer in a mammal having a Ras associated cancer; and
(ii) administering to said mammal an effective amount of a compound of Formula I: 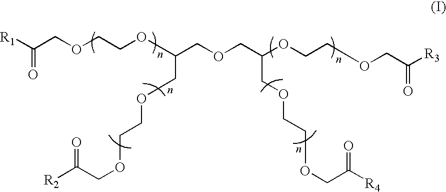
wherein
R₁, R₂, R₃ and R₄ are independently OH or 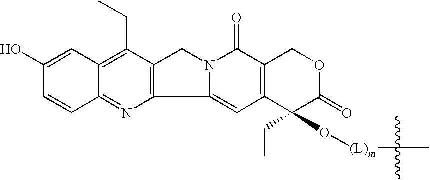
wherein
L is a bifunctional linker, and each L is the same or different when (m) is equal to or greater than 2;
(m) is 0 or a positive integer; and
(n) is a positive integer;
provided that R₁, R₂, R₃ and R₄ are not all OH,
or a pharmaceutically acceptable salt thereof.

2. The method of claim 1, wherein L is an amino acid or amino acid derivative, and the amino acid derivative is chosen from 2-aminoadipic acid, 3-aminoadipic acid, beta-alanine, beta-aminopropionic acid, 2-aminobutyric acid, 4-aminobutyric acid, piperidinic acid, 6-aminocaproic acid, 2-aminoheptanoic acid, 2-aminoisobutyric acid, 3-aminoisobutyric acid, 2-aminopimelic acid, 2,4-aminobutyric acid, desmosine, 2,2-diaminopimelic acid, 2,3-diaminopropionic acid, n-ethylglycine, N-ethylasparagine, 3-hydroxyproline, 4-hydroxyproline, isodesmosine, allo-isoleucine, N-methylglycine, sarcosine, N-methylisoleucine, 6-N-methyllysine, N-methylvaline, norvaline, norleucine, and ornithine.

3. The method of claim 1, wherein L is glycine, alanine, methionine or sarcosine.

4. The method of claim 4, wherein L is glycine.

5. The method of claim 1, wherein L is selected from the group consisting of 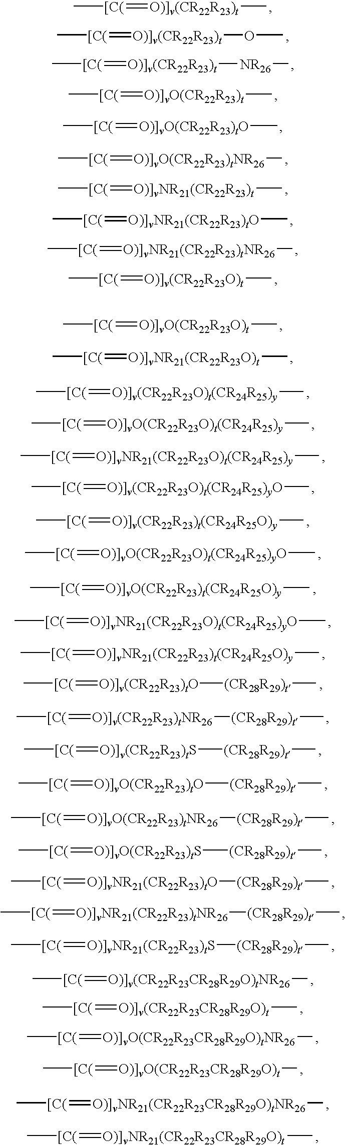 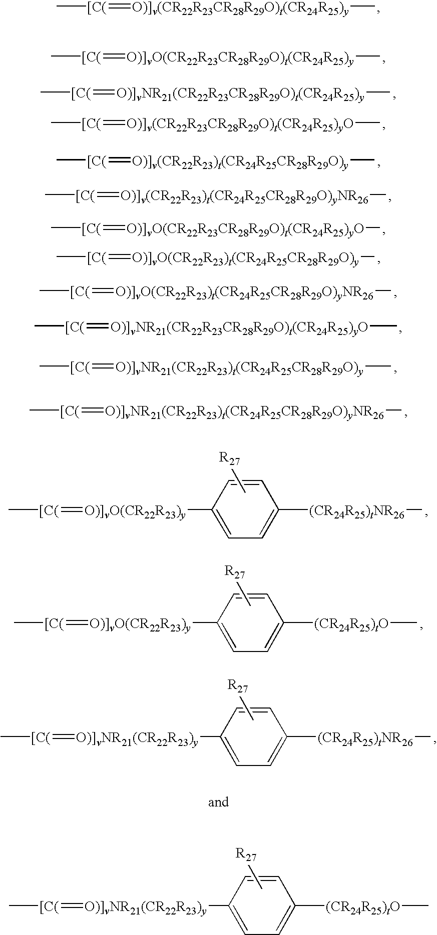
wherein:
R₂₁-R₂₉ are independently selected from the group consisting of hydrogen, amino, substituted amino, azido, carboxy, cyano, halo, hydroxyl, nitro, silyl ether, sulfonyl, mercapto, C₁₋₆ alkylmercapto, arylmercapto, substituted arylmercapto, substituted C₁₋₆ alkylthio, C₁₋₆ alkyls, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆, heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, and substituted arylcarbonyloxy;
(t), (t′) and (y) are independently zero or a positive integer; and
(v) is 0 or 1.

6. The method of claim 1, wherein (m) is from about 1 to about 10.

7. The method of claim 1, wherein (n) is from about 28 to about 341.

8. The method of claim 8, wherein (n) is from about 114 to about 239.

9. The method of claim 8, wherein (n) is about 239.

10. The method of claim 1, wherein the compound of Formula (I) is part of a pharmaceutical composition, and R₁, R₂, R₃ and R₄ are all:

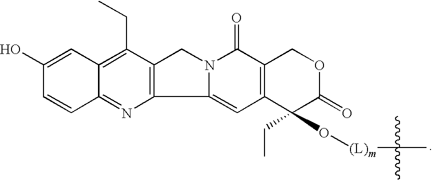

11. The method of claim 1, wherein the compound of Formula (I) is chosen from:

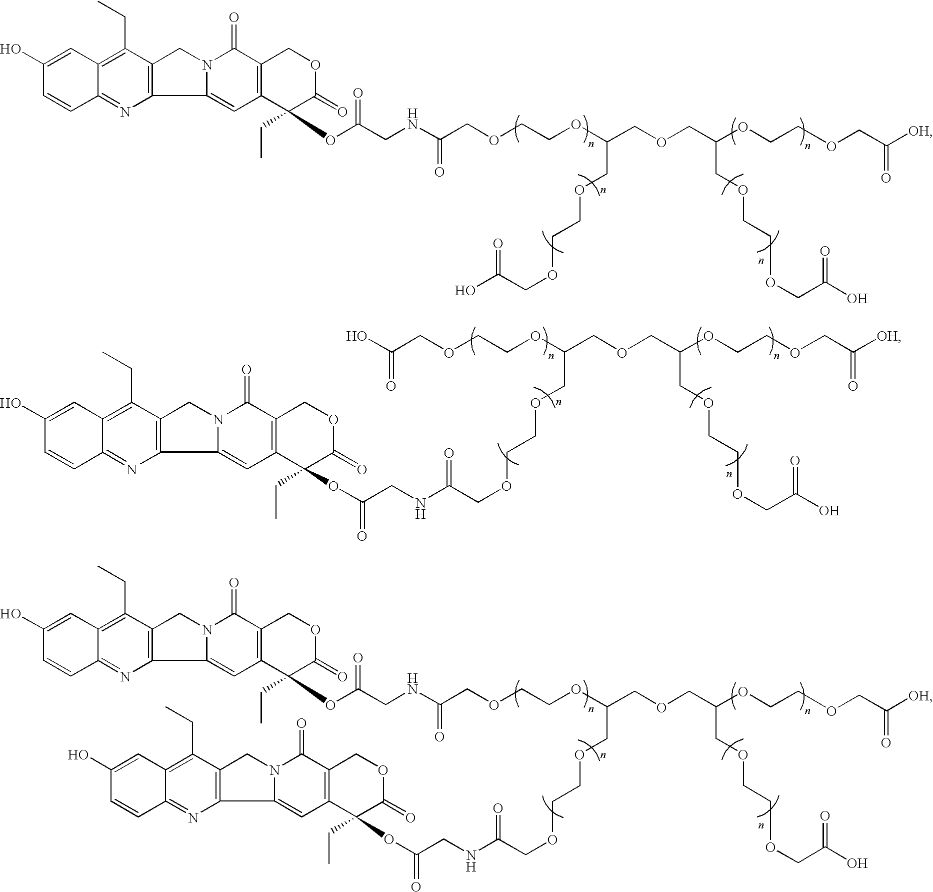

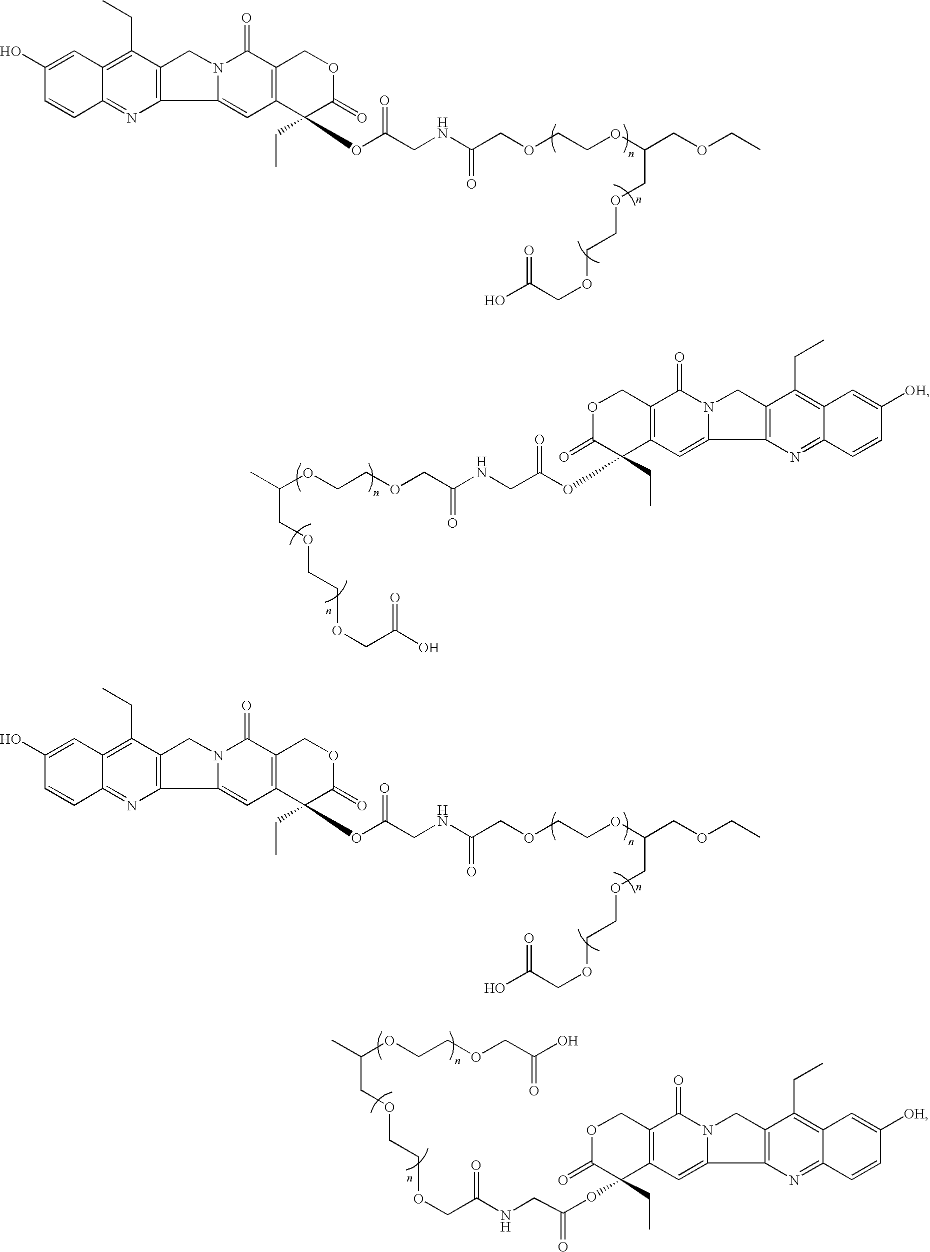

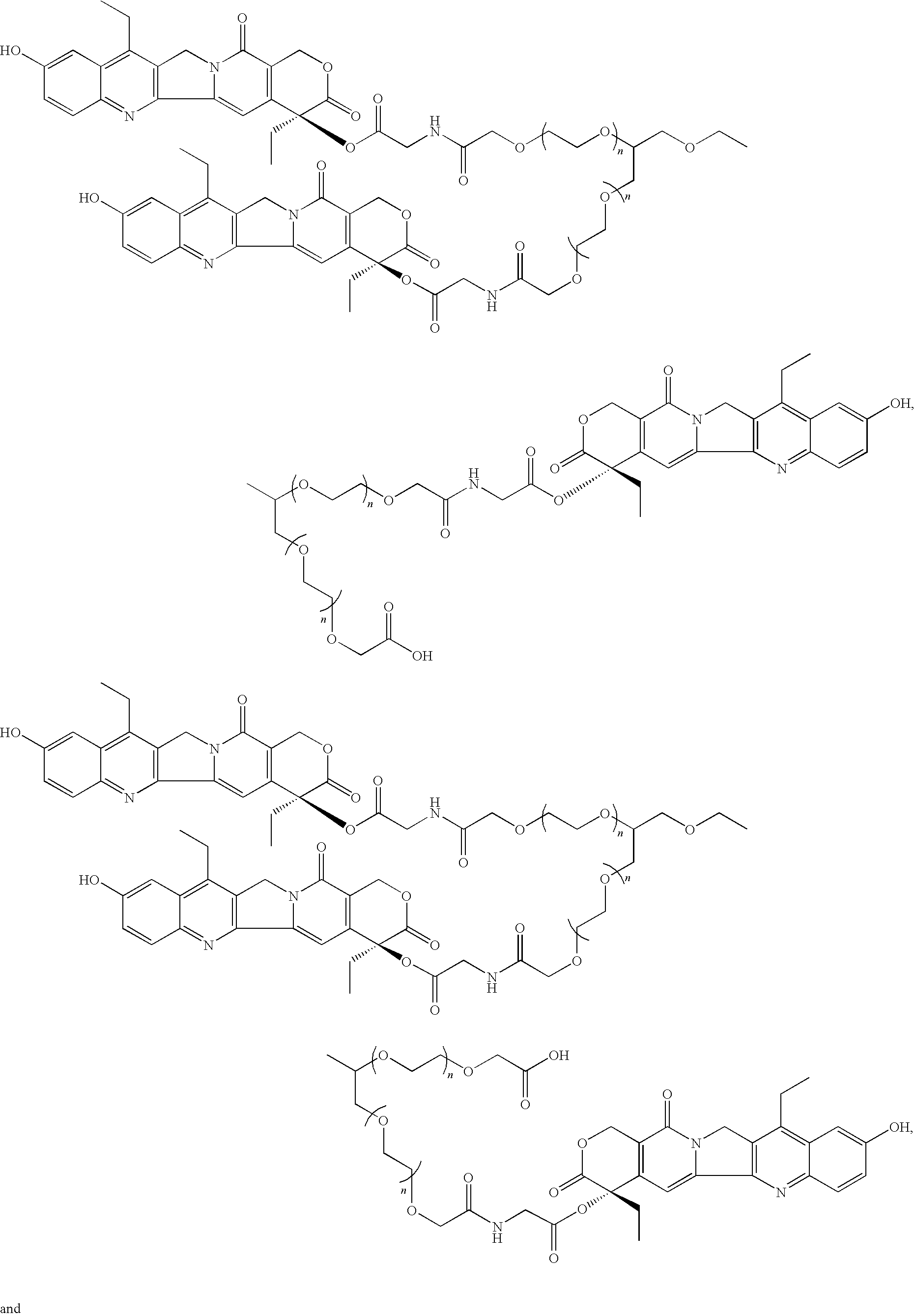

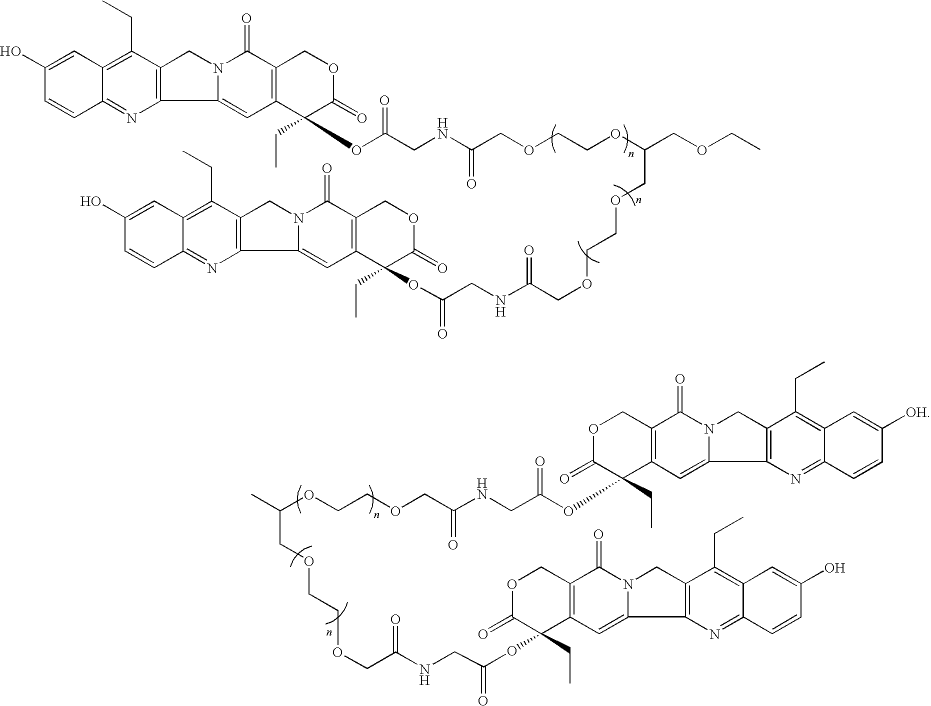

12. The method of claim 1, wherein the compound of Formula (I) is

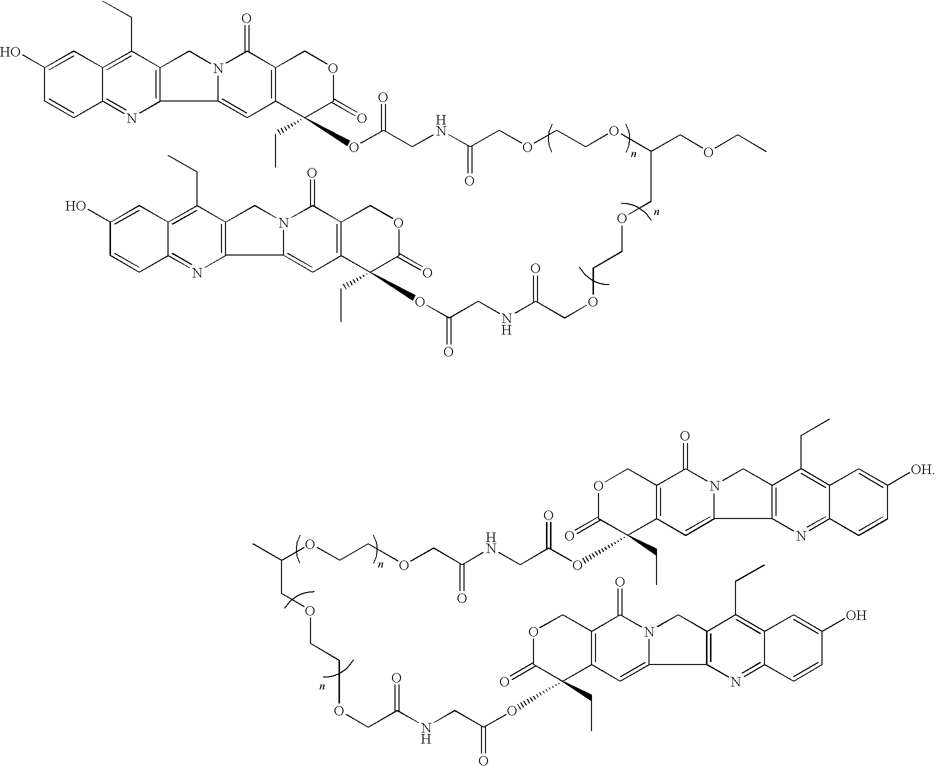

13. The method of claim 1, wherein said determining of the presence of the mutation in the RAS gene in the mammal having cancer comprises
comparing nucleic acids encoding a Ras protein isolated from the mammal to nucleic acids encoding a wild-type Ras protein.

14. The method of claim 1, wherein the Ras protein is chosen from a Kras protein, a Hras protein, and a Nras protein.

15. The method of claim 1, wherein the Ras protein is Kras2b or Kras2a,

16. The method of claim 1, wherein the mutation in the RAS gene is identified in a nucleic acid region encoding amino acids 1 to 165 of SEQ ID NO: 1, SEQ ID NO: 2, SEQ ID NO: 3 or SEQ ID NO: 4.

17. The method of claim 16, wherein the mutation in the Ras protein includes an amino acid substitution or insertion at one or more amino acid positions of 10, 12, 13, 59, 61, 117, 146 or a combination thereof.

18. The method of claim 16, wherein the substitution is at amino acid positions 12, 61 or a combination thereof of SEQ ID NO: 1 or SEQ ID NO: 2.

19. The method of claim 1, wherein said determining of the presence of the mutation in the gene encoding a Ras protein in the cancer comprises detecting SNPs associated with a known mutant RAS gene in said cancer.

20. The method of claim 1, wherein the mammal having the mutation in the RAS gene has a cancer chosen from solid tumors, colorectal cancer, pancreatic cancer, lung cancer, small cell lung cancer, stomach cancer, lymphomas, acute lymphocytic leukemia (ALL), melanoma, acute myeloid leukemia (AML), breast cancer, bladder cancer, glioblastoma, ovarian cancer, non-Hodgkin's lymphoma, anal cancer, head and neck cancer.

21. The method of claim 1, wherein the Ras associated cancer comprises metastatic cancer.

22. The method of claim 1, wherein the Ras associated cancer is resistant or refractory to an anti-cancer agent that is not a compound of Formula I.

23. The method of claim 22, wherein the Ras associated cancer is resistant to an anti-cancer agent that is chosen from camptothecin, CPT-11, an epidermal growth factor receptor antagonist, and combinations thereof.

24. The method of claim 22, wherein the epidermal growth factor receptor antagonist is cetuximab.

25. The method of claim 20, wherein the Ras associated cancer is lung cancer, colorectal cancer or pancreatic cancer.

26. The method of claim 1, wherein the compound of Formula I is administered in amounts of from about 0.5 mg/m² body surface/dose to about 50 mg/m² body surface/dose.

27. The method of claim 1, wherein the compound of Formula I is administered in amounts of from about 1 mg/m² body surface/dose to about 18 mg/m² body surface/dose.

28. The method of claim 1, wherein the compound of Formula I is administered according to a protocol of from about 1.25 mg/m² body surface/dose to about 16.5 mg/m² body surface/dose given weekly for three weeks, followed by 1 week without treatment.

29. The method of claim 28, wherein the amount administered weekly is about 5 mg/m² body surface/dose.

30. The method of claim 1, wherein the mammal is a human.

31. A method of treating a Ras-associated cancer in a mammal, comprising administering an effective amount of a compound of Formula I to said mammal: 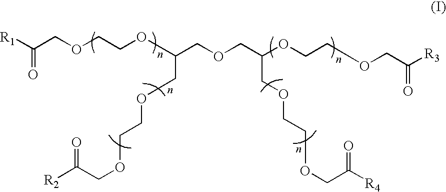
wherein
R₁, R₂, R₃ and R₄ are independently OH or 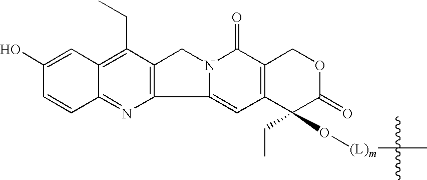
wherein
L is a bifunctional linker, and each L is the same or different when (m) is equal to or greater than 2,
(m) is 0 or a positive integer; and
(n) is a positive integer;
provided that R₁, R₂, R₃ and R₄ are not all OH;
or a pharmaceutically acceptable salt thereof;
wherein the cancer is a Ras associated cancer comprising an activated Ras protein.

32. A method of treating a Ras associated cancer in a mammal, comprising administering to said mammal a polymeric conjugate of a 7-ethyl-10-hydroxycamptothecin or a pharmaceutically acceptable salt thereof.

33. The method of claim 32, wherein the polymeric conjugate is a compound of Formula II or Formula III: 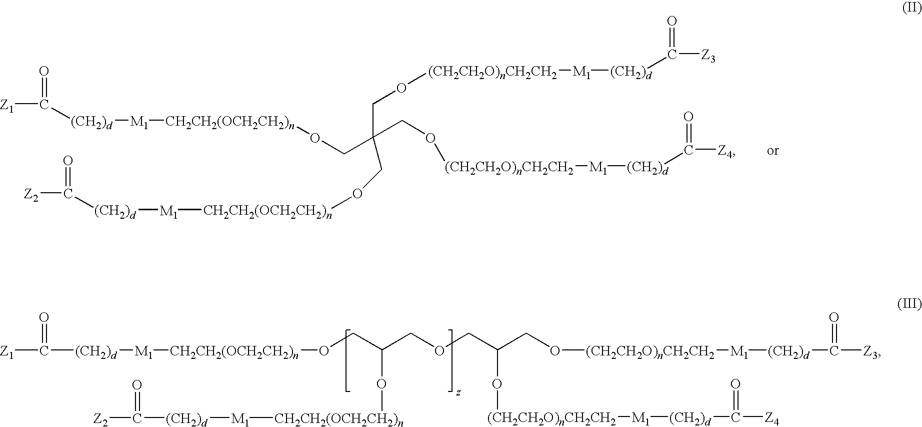
wherein
Z₁, Z₂, Z₃ and Z₄ are independently OH or (L)ₘ-D;
L is a bifunctional linker;
D is 7-ethyl-10-hydroxycamptothecin;
M₁ is O, S, or NH;
(d) is zero or a positive integer of from about 1 to about 10;
(z) is zero or a positive integer of from 1 to about 29;
(m) is 0 or a positive integer; and
(n) is a positive integer of from about 10 to about 2,300 so that the polymeric portion of the compound has the total number average molecular weight of from about 2,000 to about 100,000 daltons,
provided that Z₁ Z₂, Z₃ and Z₄ are not all OH.


SEQUENCE LISTING (7 sequences)

SEQ ID NO: 1 - protein, 189 aa, human [sequence omitted]

SEQ ID NO: 2 - protein, 188 aa, human [sequence omitted]

SEQ ID NO: 3 - protein, 189 aa, human
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 4 - protein, 189 aa, human
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 5 - DNA, 567 bp, human
atgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaa

SEQ ID NO: 6 - DNA, 570 bp, human
atgacggaatataagctggtggtggtgggcgccggcggtgtgggcaagagtgcgctgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcctga

SEQ ID NO: 7 - DNA, 570 bp, human; identical to SEQ ID NO: 6